(19) United States
(12) Patent Application Publication
(10) Pub. No.: US 2017/0007824 A1
(43) Pub. Date: Jan. 12, 2017

(54) MINIMALLY INVASIVE SPLAYING MICROFIBER ELECTRODE ARRAY AND METHODS OF FABRICATING AND IMPLANTING THE SAME

(71) Applicant: TRUSTEES OF BOSTON UNIVERSITY, Boston, MA (US)

(72) Inventors: Timothy James GARDNER, Boston, MA (US); William LIBERTI, Boston, MA (US); Jeffrey MARKOWITZ, Brookline, MA (US); Grigori GUITCHOUNTS, Boston, MA (US)

(21) Appl. No.: 14/902,734

(22) PCT Filed: Jul. 7, 2014
(86) PCT No.: PCT/US14/45584

§ 371 Date: Jan. 4, 2016

Related U.S. Application Data
(60) Provisional application No. 61/843,124, filed on Jul. 5, 2013.

(51) Int. Cl. A61N 1/05; A61B 5/04

(52) U.S. Cl. CPC A61N 1/0529; A61B 5/04001; A61N 1/0534; A61B 2562/0209; A61B 2562/125

(57) ABSTRACT

An electrode array having a splayable bundle of fibers having heat-sharpened tips. A method of manufacturing an electrode array including heat-sharpening a tip of each of a plurality of fibers; and bundling the plurality of fibers. A method of implanting an electrode array into a subject, the electrode array having a bundle of fibers, the method including exposing a target in the subject for the electrode array; and inserting the bundle of fibers into the target, where forces holding the bundle of fibers together are released during the insertion thus resulting in splaying of the fibers. An electrical connection with the fibers can be formed by a conductive material, or in high-channel count designs formed by surface mounting two-dimensional amplifier arrays to a base of a fiber array.

### CROSS-REFERENCE TO RELATED APPLICATIONS

This application claims benefit under 35 U.S.C. §119(e) of the U.S. Provisional Application No. 61/843,124, filed Jul. 5, 2013, the contents of which are incorporated herein by reference in its entirety.

### TECHNICAL FIELD

The present invention relates to a micro-electrode array, a method of manufacturing the same, and a method of implanting the same. The electrode array can utilize splaying or splayable microfibers. The electrode array can utilize carbon fiber. The electrode array can be used for chronic neural recordings for human brain-machine interfaces, for deep brain stimulating therapy, for stimulating and/or recording of peripheral nerves, for example to diagnose and/or treat medical conditions, for cochlear implants, for chronic neural recording for basic neuroscience research in animals, for chronic neural stimulation for basic neuroscience research in animals, for chronic monitoring of brain chemistry through fast scan cyclic voltammetry and the like.

### SUMMARY OF THE INVENTION

The present invention is directed to a minimally invasive electrode array that enables long term recording of brain activity, with single cell resolution including methods of fabricating and implanting the array into a subject. Multielectrode arrays are an essential tool in experimental neuroscience, yet current arrays are severely limited by a mismatch between large or stiff electrodes and the fragile environment of the brain. Chronically implanted prior art electrodes can cause ongoing damage to the brain, and an active process of rejection eventually silences neural signals. Failure of chronic prior art implants over long time-scales makes it very challenging to study the neural basis of learning, and prohibits the implementation of long term stable brain machine interfaces for human patients. To minimize electrode damage, the size of implants must be reduced, but multichannel arrays built from the smallest electrodes are impossible to implant due to buckling of the individual fibers.

The proposed electrode array solves this mechanical problem—achieving large channel count and sub-cellular (less than or equal to about 5 microns) individual electrode size in a bundle that provides mutual support for each fiber. During implantation, however, the bundle splays apart and each fiber follows its own separate course into the brain, preserving the minimally invasive properties of the single fibers. Chronic recordings from prototype designs reveal stable signals, including multiunit recordings with time-scales of months that show minimal drift in neural firing patterns. Each of the individual electrodes can be individually addressable to enable separate signals to be sent to and/or received from each electrode.

One goal of intracranial Brain Computer Interfaces (BCIs) is to restore movement and communication to people whose interaction with the physical world is either completely eliminated (as in certain forms of brainstem stroke or Amyotrophic Lateral Sclerosis (ALS)), or impaired due to spinal cord injury or amputation. Noninvasive Brain Computer Interfaces are limited by low information rates due to the poor spatial and temporal resolution of signals accessible on the scalp (outside the cranial cavity). Invasive, intracranial implants hold the potential to provide high information rates necessary to control robotic arms with precise control in amputees. The principle is demonstrated through primate experiments in which robotic limbs are controlled by implanted electrodes.

Existing brain-machine interfaces are unstable: over time, chronically implanted electrodes are encapsulated by an immune reaction that kills neurons and silences usable signals. Intracranial BCIs have not lived up to expectations of funding agencies and the general public, and a viable commercial market has not materialized, principally for lack of long term stability in neural signals. An emerging consensus among neural engineers is that the best way to avoid the reactive tissue response is to minimize the cross-section of the electrode implant. However, existing electrode technologies are limited by the underlying manufacturing methods.

The present invention is directed to a novel, ultra-small scale electrode array. One general feature of the present invention is that the individual fibers in the electrode array can spread apart or splay after implantation. This spreading or splaying reduces the stiffness of the implant and can allow brain tissue to grow between the fibers. This feature stabilizes the connection to the brain, reducing chronic damage of the tissue and preserving neural signals over long time-scales.

The present invention can include an electrode array where individual fibers are much smaller and more flexible than electrodes currently in use. In some embodiments, the bundle or array is not glued together and fibers are not twisted together as is in prior designs. In the present invention, the bundle can be held together by surface tension, a feature made possible by the small diameter and uniform geometry of the carbon fiber.

In some embodiments, individual fibers can be heat sharpened and de-insulated in a novel underwater burning process.

The present invention provides numerous advantages over the prior art. For example, the electrode array can evade immune rejection due to its relatively small size. Also, the electrode array can allow for enhanced spike sorting and signal processing due to its tight bundled geometry. Further, the electrode can provide high signal amplitudes due to a novel tip geometry provided by a new underwater heat-polishing process. Still further, the electrode can allow cells and possibly blood vessels to grow between electrode contacts, allowing for healthier tissue over chronic time-scales.

The regular geometry and material properties of carbon fibers facilitate electrode construction, which can involve a liquid interface assembly.

In some embodiments, the combination of novel materials and novel fabrication processes can provide an electrode array assembled from individual fibers as small as 4 microns. In some embodiments, each of the individual fibers can remain free to move independently after implantation, for example, in the brain. In some embodiments, the liquid interface fabrication process can be applied to other materials that have material properties comparable to carbon fibers.

In some embodiments, individual fibers in an electrode array can spread apart or otherwise shift after implantation. This reduces the stiffness of the implant, and also allows brain tissue to grow between the fibers. Both of these factors stabilize the connection to the brain, reducing chronic damage to the tissue and preserving neural signals over long time-scales.

In some embodiments, a novel method for electrode tip preparation at an air-liquid interface can provide a high-throughput process that generates reliable recording tips.

In some embodiments, arrays built from larger fibers can be chemically etched to a smaller target diameter.

In some embodiments, electrode materials with physical properties similar to carbon that are amenable to the same fabrication process can be utilized.

In some embodiments, insulation involving Parylene can be used.

In some embodiments, insulation involving silicone carbide or other materials can be used.

In some embodiments, electrode tip treatments can involve conducting polymers, gold and the like.

In some embodiments, different methods of connectorizing the electrodes can be used.

The present invention has multiple applications including but not limited to neural recording (in general), chronic neural recordings for human brain-machine interfaces, deep brain stimulating therapy, stimulating and/or recording of peripheral nerves, for example, to diagnose and/or treat medical conditions, cochlear implants, chronic neural recording for basic neuroscience research in animals, chronic neural stimulation for basic neuroscience research in animals, chronic monitoring of brain chemistry through fast scan cyclic voltammetry and the like.

The present invention is also directed to a method of fabricating carbon nanofiber probes. The fabricating method can include burning carbon fibers underwater, can include using surface tension of water (or any suitable liquid) to bundle fibers together, can include a process of burning fibers that extent above the water down to an air/water interface, can include drawing a carbon fiber bundle downward underwater, and can include turning the bundle over, e.g., rotating the bundle 180 degrees, before pulling the bundle out of the water. The carbon fibers can be oriented while being removed from the water such that the fibers are drawn together into a single bundle.

The present invention can also be directed to splaying or spreading out one or more elements of a carbon fiber bundle after implantation into, e.g., cortical tissue.

In one aspect, provided herein is an electrode array comprising a bundle of individually addressable, insulated micro-fibers with uninsulated, exposed tips, wherein the bundle of micro-fibers splay apart during implantation.

In one embodiment of this aspect, the micro-fibers comprise carbon.

In another embodiment of this aspect, the bundle is held together by van der Waals forces, and not bound together by any other material such as an adhesive.

In another embodiment of this aspect, the micro-fibers comprise a conductive, memoryless material having material properties amenable to splaying during implant.

In another embodiment of this aspect, the exposed tips are sharpened by heating.

In another embodiment of this aspect, the exposed tips are prepared by blunt cutting or by use of a focused ion beam.

In another embodiment of this aspect, the tips are heat-sharpened at an air-liquid interface.

In another embodiment of this aspect, the electrode array comprises: a micro-channel block comprising one or more openings extending through the block, wherein one or more of the micro-fibers extend through one or more of the openings.

In another embodiment of this aspect, the block comprises plastic or another machineable material.

In another embodiment of this aspect, the block is formed by a 3D printing process.

In another embodiment of this aspect, the block comprises: a main body; a pair of arms extending from the main body; and a funnel suspended by the pair of arms, wherein the micro-fibers pass through the funnel.

In another embodiment of this aspect, the funnel comprises an aperture having a diameter in a range from 100 microns to 500 microns or more.

In another embodiment of this aspect, each of the micro-fibers has a diameter of about 3-10 microns.

In another embodiment of this aspect, the diameter of each micro-fiber electrode is about 4.5 microns.

In another embodiment of this aspect, the insulated micro-fibers are insulated with parylene deposited on each of the micro-fibers at a thickness of about 1-3 microns.

In another embodiment of this aspect, one or more of the openings in the block can be filled with a conductive material to provide electrical contact between the micro-fibers and an electrical connector.

In another embodiment of this aspect, the tips are heat-sharpened with a gas/oxygen torch.

In another embodiment of this aspect, the impedance of the heat-sharpened tips is in a range of 0.1-1.5 MΩ.

In another embodiment of this aspect, the average impedance is about 1.2 MΩ.

In another embodiment of this aspect, the bundle of micro-fibers has an overall diameter of about 26 microns for a 16-channel device, about 36 microns for a 32-channel device, and about 50 microns for a 64-channel device.

In another embodiment of this aspect, each of the micro-fibers can have an exposed tip having a length of about 30-120 microns.

In another embodiment of this aspect, the length of the exposed tip can be about 89 microns.

In another embodiment of this aspect, the bundle of micro-fibers can be adapted to splay during implantation into a subject.

In another embodiment of this aspect, the electrode array yields stable signals over a time period of greater than a week.

In another embodiment of this aspect, the time period is greater than a month.

In another aspect, provided herein is a method of manufacturing an electrode array comprising: bundling a plurality of individually addressable, insulated micro-fibers; and exposing a tip of each of the plurality of insulated micro-fibers by heat-sharpening at an air-liquid interface to remove the insulation.

In one embodiment of this aspect, the micro-fibers can include carbon.

In another embodiment of this aspect, the bundle is held together by van der Waals forces, and not bound together by any other material such as an adhesive.

In another embodiment of this aspect, the micro-fibers comprise a conductive, memoryless material having material properties amenable to splaying during implant.

In another embodiment of this aspect, the method comprises: heat-sharpening tips of the plurality of micro-fibers.

In another embodiment of this aspect, the method comprises: lowering the electrode array into a liquid bath with tips of the plurality of fibers protruding above a surface of the liquid bath; and applying heat from a heat source to the plurality of fibers protruding above the surface of the liquid bath thus burning the plurality of fibers down to a surface of the liquid bath and forming an uninsulated, sharpened or tapered tip from each of the plurality of fibers.

In another embodiment of this aspect, the method comprises: raising the electrode array from a liquid bath with tips of the plurality of fibers initially pointing downward into the liquid bath; and bundling the plurality of fibers with surface tension acting on the plurality of fibers as the electrode array is removed from the liquid bath.

In another embodiment of this aspect, the method comprises: passing the plurality of fibers through a heating means in order to expose connector-side ends of the plurality of fibers.

In another embodiment of this aspect, the method comprises: filling the plurality of openings with a conductive material.

In another embodiment of this aspect, the method comprises: forming a block comprising a plurality of openings through the block; and threading each of the plurality of fibers through one of the plurality openings in the block.

In another embodiment of this aspect, the method comprises: passing the plurality of fibers through a funnel suspended from a main body of the block in order to bundle the plurality of fibers.

In another embodiment of this aspect, micro-fibers of multiple lengths are prepared by holding the electrode array at an angle relative to a liquid surface during the heat-sharpening process.

In another aspect, provided herein is a method of implanting an electrode array into a subject, the electrode array comprising a splayable bundle of individually addressable, insulated micro-fibers with uninsulated, exposed tips, the method comprising: exposing a target area in the subject; and inserting the bundle of micro-fibers into the target area, wherein forces holding the bundle of micro-fibers together are released during the insertion, resulting in micro-fibers splaying as they move into the target area.

In one embodiment of this aspect, the micro-fibers comprise carbon.

In one embodiment of this aspect, the forces are van der Waals forces, and wherein the micro-fibers are not bound together by any other material such as an adhesive.

In one embodiment of this aspect, the micro-fibers comprise a conductive, memoryless material having material properties amenable to splaying during implant.

In one embodiment of this aspect, the micro-fibers splay over a distance of about 300 μm at a depth of about 2 mm into the subject.

In one embodiment of this aspect, a degree of splaying is increased by a lateral tension held in the micro-fibers during the inserting step.

In one embodiment of this aspect, a degree of splaying is limited by partially gluing micro-fibers together before the inserting step, allowing an end of the bundle to splay while a body of the bundle does not splay.

In one embodiment of this aspect, the splayed array of micro-fibers forms a predefined geometric shape that can be controlled by using micro-fibers of multiple lengths in a single bundle.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification and the spirit and scope of the teachings herein.

In the drawings, where like reference numerals refer to like features in the specification:

FIG. 1 generally depicts an array assembly;

FIG. 1A depicts a 3D-printed plastic block with wells for 16 fibers, where the fibers are heated by passing them through a gas/oxygen torch and where the wells are filled with conductive material such as silver paint or metallic adhesive;

FIG. 1B generally depicts a process for heat-sharpening of electrode tips;

FIG. 1B1, upper left side, depicts an assembled array lowered into a water bath with the tips of the carbon fibers protruding above the surface of the water;

FIG. 1B1, upper right side, depicts the assembled array after a gas/oxygen torch is passed over the surface of the water, thus burning the carbon and the insulating Parylene down to the water surface;

FIG. 1B1, lower left side, is an SEM image of a blunt cut carbon fiber electrode, with insulating frayed near the tip;

FIG. 1B1, lower right side, is an SEM image of the carbon fiber electrode after passing the torch over the exposed tips, which shows that the carbon fiber tapers to a sharp point;

FIG. 1B2, upper left side, depicts the array as it is being taken out of the water with the tips pointing down;

FIG. 1B2, upper right side, depicts the array after it is taken out of the water with the tips pointing down and shows how surface tension acts to bring the carbon fibers into a single tight bundle;

FIG. 1B2, lower side, shows four sequential side views of the tip of the array as surface tension acts to bring the carbon fibers into a single tight bundle;

FIG. 1B3 is a chart of impedance of the array before and after torching;

FIG. 1C depicts an assembled array with close-up views of a central portion of the bundle (lower left side) and a portion near the tip of the bundle (lower right side);

FIG. 2A is a histogram of the heat-sharpened pre-implant electrode impedance;

FIG. 2B is a chart of impedance of fibers in 7 implanted arrays measured at various time points after implanting;

FIG. 3 depicts a single unit recording in a singing bird;

FIG. 4 depicts chronic recording stability in the singing bird;

FIG. 5 depicts a principal cell recorded in HVC;

FIG. 6 depicts simultaneously recorded activity;

FIG. 7 depicts an example of stability of spike features in rigorous single units;

FIG. 8 depicts stability of sorted multi-units and single-units;

FIG. 9A depicts a carbon fiber coated in fluorescent Parylene-C, after it had been heat-sharpened in wide-field, under a UV filter and a merged image;

FIG. 9B depicts another example fiber in wide-field, under a UV filter and a merged image;

FIG. 10 generally depicts average waveforms of isolated single units recorded acutely with 16-channel carbon fiber arrays;

FIG. 10A is a time versus voltage chart for a unit recorded in auditory area Field L in an awake head-fixed bird;

FIG. 10B is a time versus voltage chart for a unit from the pre-motor nucleus HVC in an anesthetized bird;

FIG. 10C is a time versus voltage chart for an isolated unit found in the basal ganglia of an anesthetized bird;

FIG. 10D is a time versus voltage chart for a unit recorded in Field L of an awake, head-fixed bird;

FIG. 11 depicts clusters for the chronic signal shown in FIG. 3;

FIG. 12 depicts example projection neuron recordings of various signal qualities;

FIG. 13 depicts single trial voltage traces from 3 rasters shown in FIG. 12;

FIG. 14A depicts a bursting cell with high amplitude positive peaks recorded from a bird implanted in Area X;

FIG. 14B depicts a similar cell recorded from a bird implanted in HVC;

FIG. 15 generally relates to the tetrode effect;

FIG. 15A and FIG. 15C depict example traces recorded from a chronically implanted bird showing correlated signal on two channels;

FIG. 15B and FIG. 15D depict scatter plots of spike amplitudes on the two channels showing correlated signal;

FIG. 16 generally depicts sorted multi-unit stability;

FIG. 16A depicts four signals recorded in HVC on different channels in one bird;

FIG. 16B depicts principal components analysis of the firing rate patterns shown in FIG. 16A;

FIG. 17 depicts an HVC interneuron recorded on sessions 107 days apart;

FIG. 18 generally relates to single unit stability;

FIG. 18A depicts a putative HVC interneuron recorded on twelve sessions across 23 days;

FIG. 18B is a chart of Average (+SD) waveforms on the first (top), sixth (middle) and last (bottom) days;

FIG. 18C is a chart of corresponding ISI distributions;

FIG. 19 includes the results of three channels recorded from a carbon tetrode;

FIG. 20A depicts distributions for waveform, ISI, and IFR scores for stable single units (black) and the full ensemble of units recorded (gray), quantified with Jensen-Shannon Divergence;

FIG. 20B depicts decision boundaries drawn using the three measures;

FIG. 20C is a beeswarm plot of the longevity of neurons held for more than a single recording session (18/27 interneurons) according to a classifier according to the present invention;

FIG. 21 is a chart comparing the present tunneling microfiber arrays (8 data points along the left side of the chart adjacent the y-axis) having ultra-small minimum feature diameters with high channel count with the cross section (x-axis) shown in μm;

FIG. 22 shows an electrode array (SEM, three length scales left) and single electrode imaged with Anthracene doped parylene (right);

FIG. 23 shows electrode fibers (white in reverse bright-field) splayed over a distance of 300 μm at a depth of 2 mm;

FIG. 24 shows a two photon in-vivo image of a 16 channel electrode insertion in a transgenic zebra finch;

FIG. 25 depicts an embodiment of the present invention including a bundle of hundreds of electrode fibers; the figure is an illustration only;

FIG. 26 depicts an embodiment of the present invention including amplifiers formed, for example, by surface mounting a plurality of electrodes in the form of a two-dimensional array on a flexible substrate;

FIG. 27 depicts a process for heat-sharpening of electrode tips, where an array is held at different angles prior to heating;

FIG. 28 depicts the array of FIG. 27 after heating;

FIG. 29 depicts self-splaying electrodes used for recording and stimulation of a songbird hypoglossal nerve tracheo-syringeal (TS) branch;

FIG. 30 is a TS nerve cross-section;

FIG. 31 is a chart depicting 16 channel recordings of self-splaying electrodes in songbird hypoglossal nerve tracheo-syringeal (TS) branch; and

FIG. 32 is a chart depicting vocalizations evoked by TS nerve stimulation in an anesthetized zebra finch.

### DETAILED DESCRIPTION

It should be understood that this invention is not limited to the particular methodology, protocols, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities used herein should be understood as modified in all instances by the term “about.”

All publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Any known methods, devices, and materials may be used in the practice or testing of the invention, and the methods, devices, and materials disclosed herein are provided for purposes of illustration and to facilitate an understanding of the inventions.

### SOME SELECTED DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to the invention, yet open to the inclusion of unspecified elements, whether useful or not.

As used herein the term “consisting essentially of” refers to those elements useful for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal. The mammal can be a human, non-human primate, mouse, rat, dog, cat, horse, or cow, but are not limited to these examples. Mammals other than humans can be advantageously used as subjects that represent animal models of disorders.

A subject can be one who has been previously diagnosed with or identified as suffering from or having a disease or disorder caused by any microbes or pathogens described herein. By way of example only, a subject can be diagnosed with sepsis, inflammatory diseases, or infections.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

Chronically implanted microelectrodes arrays are a useful tool in fundamental neuroscience research. These devices can provide intracranial brain-machine interfaces in humans, restoring movement and communication to people whose interaction with the physical world is either completely eliminated due to stroke or disease or impaired due to spinal cord injury or amputation. Proof of principle experiments have demonstrated control of robotic limbs through intracranial electrodes, and efforts around the world are building toward a time in the near future when researchers will be able to monitor brain function with vast numbers of probes that sense neural activity with high precision, revealing principles of the human mind. However, monitoring large ensembles of neural activity in the living brain with single neuron and single spike resolution faces challenging technical limitations. For prior art microelectrodes, one of the biggest limitations of chronic neural recording is a reactive tissue response that encapsulates electrodes and kills or damages neurons. This rejection of electrode implants leads to particularly serious problems when recording from densely packed neurons in small animal research models, or when recording over the multi-year time-scales required for practical human neural prosthetics.

The present invention is directed to a novel multielectrode array that is designed to be minimally invasive, while still providing stable recordings from many neurons simultaneously. In accordance with some embodiments, the “tunneling fiber array” consists of dense bundles of ultra-small sharpened carbon fibers that can be discretely inserted into the brain. One feature of the design is observed during implantation of the electrode; rather than tearing through tissue in the un-compliant manner of existing commercial arrays, the proposed array splays during insertion and individual fibers are free to follow their own path of least resistance into the brain. Neural recordings from prototype devices are stable over a timescale of months. Electrode splaying contributes to chronic recording stability. The interface between brain tissue and electrode through in-vivo imaging and histology can be examined. The tunneling fiber array shows reduced tissue damage due to the small scale fibers and due to the ability of single electrodes to separate from each other during implant. Over long time-scales these features minimize damage to neurons and blood vessels in the space adjacent to the fibers, promoting stable recordings. The present invention can be used to enable new fundamental research that utilizes long term recordings with single cell resolution. Since the small animal model is a particularly challenging test bed for chronic recording, it is anticipated that results of the present study will also inform future designs for minimally invasive electrodes in human and animal brain machine interfaces.

The bundle can comprise two or more individually addressable insulated micro-fibers. That is, each fiber in an array can function as an individually addressable electrode than can be used to send a signal to the electrode tip (e.g. for stimulation) as well as to receive signals from cells in contact with the electrode tip (e.g. for monitoring and recording). In accordance with embodiments of the invention, each electrode in the bundle can be separately addressable. In accordance with some embodiments of the invention, two or more electrodes can be connected together and can be addressable as a single electrode.

The present inventors describe herein a fabrication method for a 16 channel electrode array consisting of carbon fibers (<5 μm diameter) individually insulated with an insulator such as Parylene-C and heat-sharpened. The diameter of the array is approximately 26 microns, along the full extent of the implant. Carbon fiber arrays were tested in HVC (used as a proper name), a song motor nucleus, of singing zebra finches where individual neurons discharge with temporally precise patterns. Previous reports of activity in this population of neurons have required the use of high impedance electrodes on movable microdrives. Here, the carbon fiber electrodes provided stable multi-unit recordings over time-scales of months. Spike-sorting indicated that the multi-unit signals were dominated by one, or a small number of cells. Stable firing patterns during singing confirmed the stability of these clusters over time-scales of months. In addition, from a total of 10 surgeries, 16 projection neurons were found. This cell type is characterized by sparse—stereotyped firing patterns, providing unambiguous confirmation of single cell recordings. Carbon fiber electrode bundles can provide a scalable solution for long-term neural recordings of densely packed neurons.

Carbon electrodes can be biocompatible and due to a thin profile, are minimally invasive upon implantation. However, practical methods for preparing electrode tips, assembling and implanting arrays have not been described in the prior art. As a result, their utility for chronic recording remains unknown. The present invention is directed to a carbon fiber electrode that can provide stable recordings from small neurons in a singing bird and other animals. The electrode includes 16-channels created by bundling together individually insulated carbon fibers (<5 μm diameter). Carbon fiber electrodes recorded approximately 5.3 cells per implant, in a fixed location. 61% of neurons were stable through one week, 33% through two weeks, and 22% through one month.

A powerful approach to the study of learning involves tracking neural firing patterns across time. Optical methods for stable recording are developing rapidly {Harvey:2012du} but the temporal resolution of electrical recordings remains unsurpassed, and chronically implanted microelectrodes are central to scientific studies of neural circuit function in behaving animals, and central to the development of intracranial brain-machine interfaces in humans {Donoghue:2008dn}. In primate motor cortex, relatively large neurons can be tracked for weeks {Tolias:2007dy, Dickey:2009wi, Fraser:2012bz} using commercially available electrode arrays. This has enabled researchers to study the stability of motor tuning {Rokni:2007fh, Chestek:2007e1} and the process of memory formation {Thompson:1990vj, Kentros:2004wq}; and it has provided the basis for brain machine interface technologies {Koralek:2012ib}.

Over time, chronically implanted prior art electrodes become severely limited by a tissue reaction that eventually encapsulates the electrode, killing neurons in the vicinity of the electrode. The limitations of this tissue response are particularly acute if the goal is recording from densely packed neurons in small brains. For a silicone array whose cross-section is 15×200 microns, histological markers of gliosis and neuron death reveal tissue damage extending up to 300 microns or more from the implant {Biran:2005dm}. This length-scale of tissue damage does not prohibit long term recording from pyramidal neurons in primate cortex whose large polarized dendrites and large somas (up to 100 microns) produce a strong signal for extracellular recording. However, the length scale of tissue damage becomes prohibitive when recording from many cell types in smaller organisms. For example, in the songbird nucleus HVC (used here as a proper name), somas are only 8-15 microns in diameter {Mooney:2005db} and closely-packed in clusters making soma-soma contact {Scott:2012we}. Dendrites in HVC are also compact (40-100 micron radius), and spherical in shape rather than polarized {Mooney:2005db, Lewicki:1996tl, Katz:1981ub}. To isolate the weak signal generated from these cells, electrodes with small recording surfaces are advanced with motorized microdrives, allowing micron-scale control over electrode positioning. The absence of any report of single neuron isolation in HVC with a fixed chronic electrode implant underscores the difficulty of recording small cells with an implant whose damage length scale is large relative to the target neurons. Examples of multi-day recordings in mouse hippocampus can be found {Kentros:2004wq, Koralek:2012ib} that employ movable tetrode microwires, but recording methods that make this process more efficient are needed. Ultimately, to facilitate long term recordings from densely packed neurons in small animals, and to improve the longevity of human and primate neural interfaces, electrode designs are needed that reduce chronic tissue damage. Increasing attention to the limitations of current microelectrode technologies has led to a strong conclusion that the cross-section of implanted electrodes must be minimized to reduce chronic disruption of the blood brain barrier {Biran:2005dm, Polikov:2005cq}. Orthogonal pressures are driving an increase in the number of recording sites per implant, but increasing the density of recording sites may be counter-productive if the implant size also increases.

Recently proposed carbon fiber “ultramicroelectrodes” promise to reduce damage upon implantation, and may partially evade immune rejection {Kozai:amAEGRtY, Kozai:2012 bp}. Glass insulated carbon fibers have been used for cyclic voltammetry and extracellular recording for some time {Garris:1994tt, ArmstrongJames:1979ue}, but one improvement of the “microthread” electrode according to the invention involves doing away with the large-diameter glass insulation in favor of a thin (1 micron) layer of parylene deposited over a small (3-10) micron diameter carbon fiber. This also serves to dramatically reduce the stiffness of the implant, another factor hypothesized to contribute to tissue damage {Subbaroyan:2005jc}. The small profile over the full length of the electrode, in principle, could provide for minimal chronic tissue damage and neuron death. The small scale and flexibility of individual carbon “microthread” electrodes according to the invention makes implantation challenging. The present invention provides a practical method for making and implanting high channel-count carbon fiber electrodes. Furthermore, carbon fiber electrodes with sharpened tips can be useful for implantation into some potential targets such as small (<300 micron), peripheral nerves.

The 16 channel carbon fiber array described here has a final cross-section of approximately 26 microns, on par with single micro-wires in commercial microwire arrays (30-50 micron wires in all Tucker-Davis microwire arrays, for example.) The principle innovations of this work arise from carbon fiber manipulations using the elements of fire, air and water: burning partially submerged carbon fibers leads to a consistent electrode tip preparation. Drawing carbon fibers through an air-liquid interface leads to surface-tension driven bundling of the fibers, resulting in an implantable array.

To test the design, the present inventors examined song coding in zebra finches—a uniquely tractable test-bed for assessing chronic electrode stability. Zebra finch song is a stereotyped natural behavior produced by a distinctive learned pattern of neural activity. As the bird sings, each of three cell types in pre-motor nucleus HVC—interneurons, basal ganglia-projecting neurons, and motor output neurons—discharge in a stereotyped, characteristic pattern with minimal spike-time jitter (<1 ms for motor output neurons) between renditions of the same song {Hahnloser:2002hj, Kozhevnikov:2007eu}.

The various cell types in HVC are small (8-15 microns), and synchronous elevated firing rates of interneurons during song make isolation of single units challenging. Current approaches to recording the three cell types in HVC require the use of high impedance electrodes positioned close to individual cells using a motorized microdrive {Fee:2001wh}. Neurons isolated in this manner in HVC are typically not recorded for a time-scale longer than tens of minutes. Using fixed implants of the carbon fiber arrays according to the invention described here, the present inventors recorded unambiguous single units in HVC over timescales of 4-12 hours, and “sorted” multi-unit clusters over time-scales of months. The present inventors find that the precise temporal patterns recorded in HVC are stable over the time-scales of the recordings. This feature provides a means of validating signal stability independently of spike waveforms, providing a useful test-bed to assess chronic recording methods.

Methods

Carbon Microthread Arrays

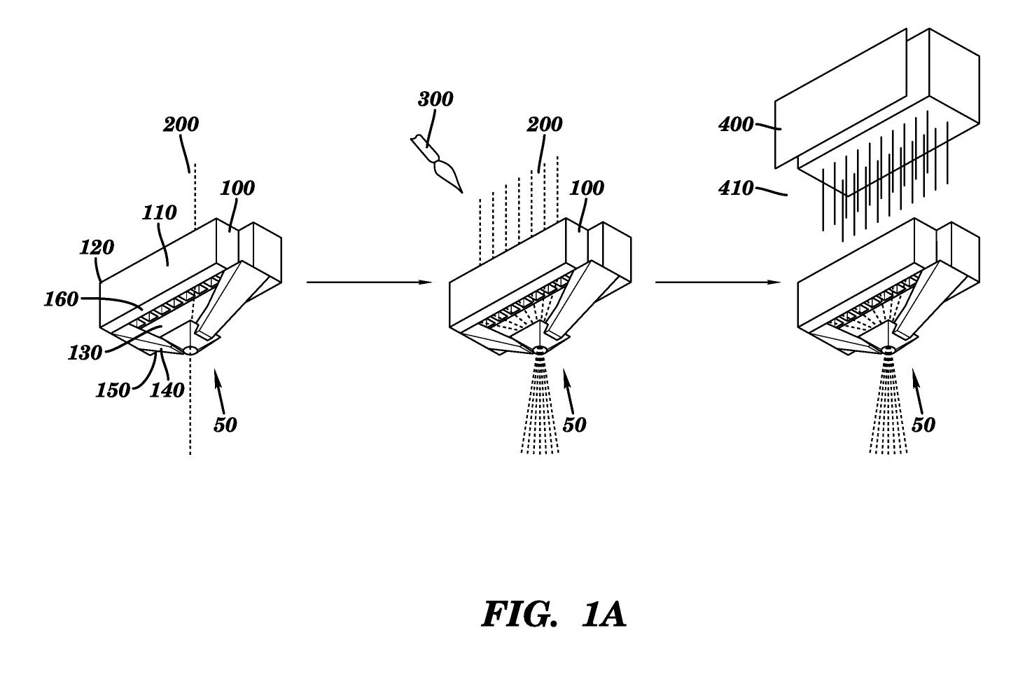

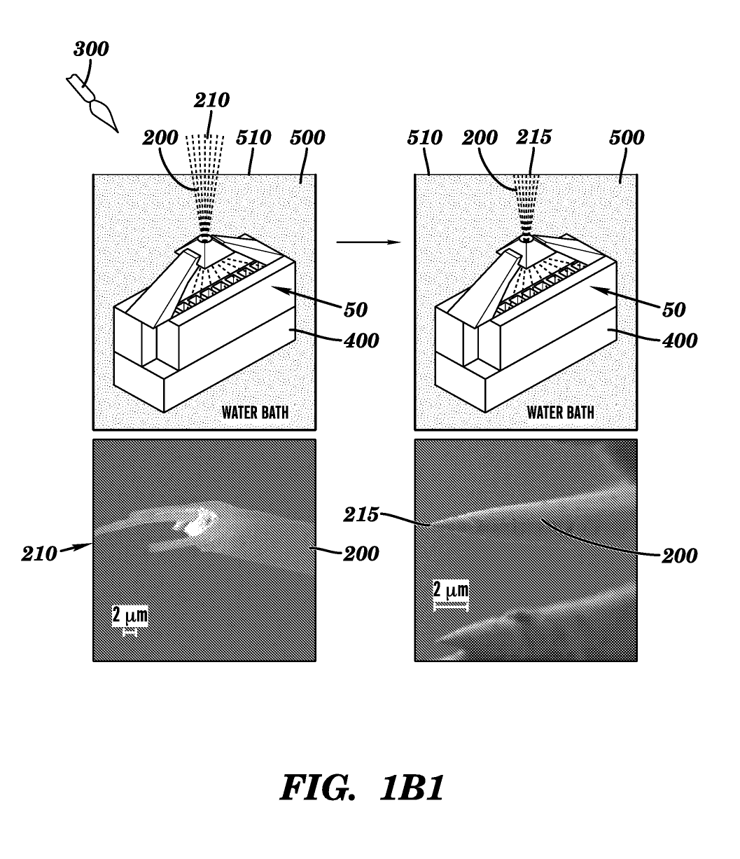

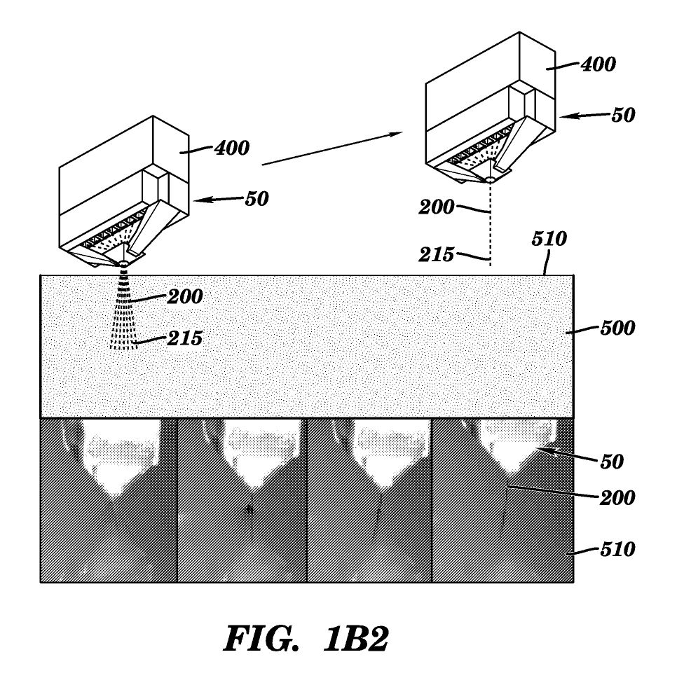

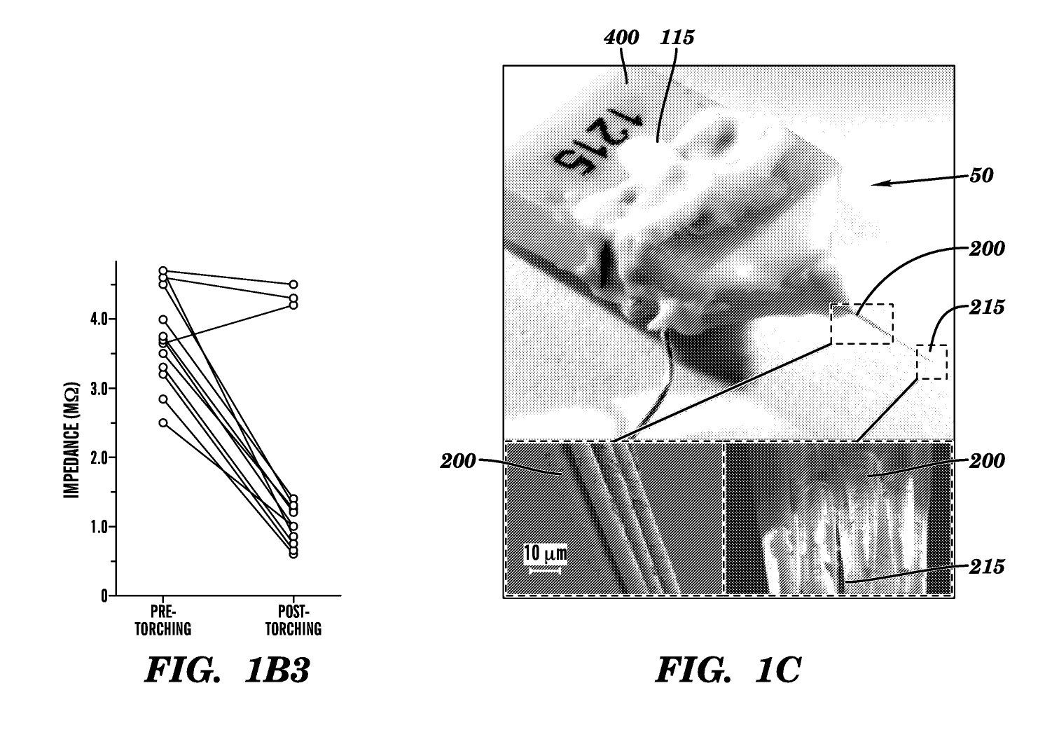

FIG. 1 generally depicts an example of assembly of an array 50 according to the present invention. FIG. 1A, left side, is a diagram of a block 100 with wells 110 for fibers 200. Each of the fibers 200 can be carbon fiber or the like. Each of the fibers 200 can have a diameter of about 3-10 microns. Each of the fibers 200 can be coated with an insulator such as a 1 micron thick layer of parylene or the like. The block 100 can be formed of plastic or any other suitable material, which may have an insulating property. The block 100 can be formed using any suitable manufacturing process such as molding, casting, machining and a 3D printing process. As shown in FIG. 1A, each well 110 extends through a main body 120 of the block 100 so as to have an opening at each end of the well 110, one opening on top of the main body 120 (not shown) and another opening at the bottom of the main body 120 (shown). Fibers 200 can be threaded through the wells 110 on the top of the block 100 and exit through the hole on the bottom. In some embodiments, the block 100 can have a pair of extending arms 130 supporting a central portion 140 having an opening 150 through the block 100. The central portion 140 can also be referred to as a multi-channel funnel. It is noted that the arms 130 and the central portion 140 are optional and not required. When provided, any other suitable shape of the central portion 140 can be employed so long as it facilitates the function of the present splayable electrode microfibers. In some embodiments, the central portion 140 can facilitate collection and support of the fibers 200 into a bundle, which is initially splayed as shown in FIG. 1A, middle, and FIG. 1A, right side. Each of the arms 130 can extend from either side of the main body 120 of the block 100 to the central portion 140 so as to have an opening between the bottom of the main body 120 and the central portion 140. In some embodiments, the block 100 can have as many as 16 or more wells 110 for up to 16 or more fibers 200; however, any suitable number of wells 110 and fibers 200 can be provided. In some embodiments, it is not necessary for each well 110 to include a fiber 200 and some wells 110 can include more than one fiber 200.

As shown in FIG. 1A, middle, in order to expose the connector-side ends of the fibers 200 from parylene, the fibers can be heated by passing them through heat generated by a heating source 300 such as a gas/oxygen torch. The heating source 300 is not limited to the gas/oxygen torch but can be any suitable heating source. In some embodiments, the heating source can generate enough heat to remove the insulator while providing desirable heat-polishing or heat-sharpening to the fibers 200. For example, when the insulator comprises parylene and the fibers 200 comprise carbon fiber, the heating source should generate enough heat to remove the parylene, i.e., greater than about 290° C., and should achieve a temperature that is beneficial to the performance of carbon fibers as electrodes, i.e., on the order of at least about 1,000° C. For example, when a gas/oxygen torch is used, the torch can generate temperatures of about 2,000° C. to about 3,500° C. (depending on the type of gas used), which is found to be useful for heat-polishing or heat-sharpening the fibers 200 according to the present invention.

Each of the wells 110 can be filled with a conductive material such as silver paint 115 (see FIG. 1C) to make electric contact with a suitable connector 400. For example, as shown in FIG. 1A, right side, a suitable connector 400, such as an Omnetics connector, can be used to make electrical contact with the array 50. The pins 410 of the connector 400 can slide into the wells 110 in the block 100. For example, when using the Omnetics connector A79038-001, 4 of the 20 pins, two on each side, are non-functional and can be used for guide posts or keys, leaving 16 pins for engagement with the 16 fibers 200 at a junction inside each of the 16 wells 110, respectively.

FIG. 1B generally depicts a process for heat-sharpening of electrode tips. FIG. 1B1, upper left side, depicts an assembled array 50 lowered into a water bath 500 with the tips 210 of the carbon fibers 200 protruding above the surface 510 of the water 500. FIG. 1B1, upper right side, depicts the assembled array 50 after a gas/oxygen torch 300 is passed over the surface 510 of the water 500, thus burning the carbon and the insulating parylene of each fiber 200 down to the water surface 510. FIG. 1B1, lower left side, is an SEM image of a blunt cut carbon fiber electrode 200, with insulating frayed near the tip 210. FIG. 1B1, lower right side, is an SEM image of the carbon fiber electrode 200 after passing the torch 300 over the exposed tips 215, which shows that the carbon fiber 200 tapers to a sharp point.

FIG. 1B2, upper left side, depicts the array 50 as it is being taken out of the water 500 with the tips 215 pointing down. FIG. 1B2, upper right side, depicts the array 50 after it is taken out of the water 500 with the tips 215 pointing down and shows how surface tension acts to bring the carbon fibers 200 into a single tight bundle. FIG. 1B2, lower side, shows four sequential side views of the tip 215 of the fibers 200 of the array 50 as surface tension acts to bring the carbon fibers 200 into a single tight bundle. In other words, as the array 50 is lifted out of the water 500, surface tension acts to draw the carbon fibers 200 together into one bundle. The resulting tight geometry of the electrodes allows for recording from small brain nuclei and for sorting units based on a common signal, i.e., “tetrode” effect.

FIG. 1B3 is a chart of impedance of the array before (e.g., FIG. 1B1, lower left side) and after (e.g., FIG. 1B1, lower right side) torching. FIG. 1B3 shows that torching the carbon fibers greatly reduces impedance (tested at 1 kHz).

FIG. 1C includes three photographs of the assembled array 50. The 16-electrode bundle is approximately 26 μm in diameter (lower left). The immersion tip burning process exposes approximately 89 μm of carbon (lower right) from the end of the fiber.

In an embodiment of the present invention, 4.5-micron diameter carbon fiber threads (Goodfellow USA, Grade UMS2526) can form the basis of the array. (Young's modulus of 380 GPa compared to tungsten's 400 GPa: volume resistivity 1000 μΩcm compared with 5.4 μΩcm for tungsten.) Epoxy sizing can be removed by heating fibers at 400° C. for 6 hours {Schulte:1998fn} using a Paragon SC2 kiln (Paragon). A 1 μm layer of Parylene-C(di-chloro-di-p-xylylene) (Kisco) can then deposited using an SCS Labcoter 2 (PDS-2010, Specialty Coating Systems) using 2.3 g of parylene and factory settings as follows: Furnace, 690° C.; Chamber Gauge, 135° C.; Vaporizer, 175° C.; and Vacuum at 15 vacuum units above base pressure. The integrity of the coating was verified through bubble testing initially, and defects were never found. Fourteen coated fibers and 1 coated or uncoated fiber (for the reference) can be threaded through a custom plastic block designed in SolidWorks (Dassault Systems) and 3-D printed using stereolithography (Realize Inc.) (one channel out of the possible 16 was used for the microphone trace and one was shorted with the reference, making a total of 14 electrophysiology channels) (FIG. 1A). The block can contain 16 wells (e.g., 18×27 mils, 450×685 microns) that direct the carbon fibers through a small aperture (12 mils/305 microns in diameter) in the funnel. At this stage the carbon fibers exiting the aperture are splayed (FIG. 1A, middle).

On the top side, the block can be cut to fit the straight tails of an Omnetics connector (A79038-001, Omnetics). Fibers at the mating end of the carbon can be briefly passed through a gas/oxygen torch to remove insulation for making electrical contact (Smith Equipment) (FIG. 1A, middle). The fibers can then be connected to an Omnetics connector using conductive material such as silver paint (Silver Print part no. 842-20G, MG Chemicals), which can be spread into the wells housing the carbon fibers. The connector can be pressed into the silver-filled wells and glued to the plastic block using light bonded dental acrylic (Flow-It ALC, Pentron Clinical) (FIG. 1A, right).

Initially, the carbon fiber bundle can be blunt cut with surgical scissors (Fine Science Tools) or a razor blade to expose the insulation. This can result in widely varying impedances, often as high as 4 MΩ (measured at 1 kHz with a Bak Electronics IMP-2 impedance tester). All impedance measures reported here are in phosphate buffered saline solution (Sigma Aldrich D5773 SIGMA). The wires can be embedded in carbowax (polyethylene glycol) and can be cut using a vibratome, but this procedure was found not to effectively reduce impedance. The tips of the fibers can be ground on a spinning plate (a modified hard drive) for up to 30 minutes, but this procedure did not produce desirable impedance either.

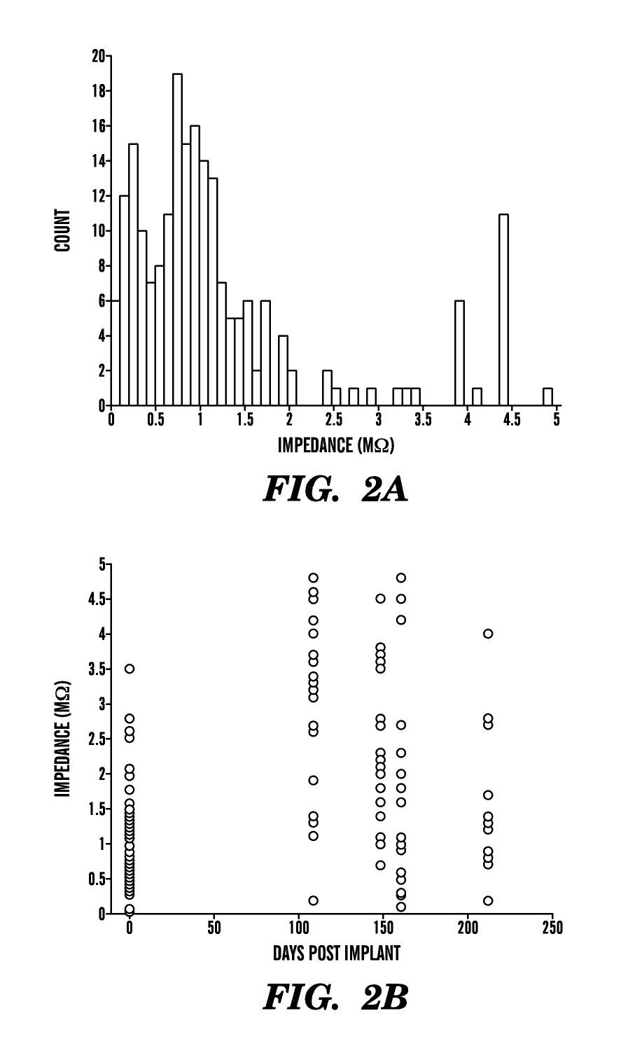

To produce a consistent low-impedance tip, according to the present invention, a process of heat-sharpening the tips with, for example, a gas/oxygen torch (flame ˜4.5 mm across, 7.5 mm in length) can be employed. The process involves holding the array underwater while burning the exposed tips above the surface of the water (FIG. 1B). With the array secured in a water bath with carbon fiber tips protruding above the surface of the water (FIG. 1B, left), the carbon was burned down to the surface of the water with the torch (FIG. 1B, middle). The water acted as a flame retardant/insulator, providing control over the amount of Parylene-C taken off of the tip of the carbon. This had two desirable effects: (1) sharpening the tip of the bundle, and (2) lowering the tip-impedance to an acceptable range for extracellular recording (FIG. 2). By comparison, the present inventors found blunt-cutting of the Parylene insulated electrodes to produce widely varying impedance values and unreliable signals. FIG. 1B1, lower left panel, shows one blunt-cut electrode, revealing a carbon recording surface recessed from the cut Parylene surface. A recent study found it necessary to surface treat parylene-insulated carbon fibers with PEDOT for recording chronic extracellular signals {Kozai:2012 bp}. The large surface area of the exposed carbon in the heat-sharpened electrode may explain why chronic signals were found in the present study without additional surface modifications required in the previous study.

In a final step, the array can be slowly drawn out of the bath with the fibers having at least a slightly downward facing orientation. In this step, surface tension pulls together the fibers into a single bundle, and this bundle remains together after the fibers dry (FIG. 1B2, upper right), allowing the entire bundle to be implanted. FIG. 1C shows an example of the (˜250 mg) assembled 16-channel array. This final weight is comparable to commercially available electrode arrays with a similar number of contacts (300 mg for a 16 channel Omnetics TDT array, 140 mg for a 16 channel H-style probe from Neuronexus, and 130 mg for a 16 channel microwire array from Microprobes). In the final construction, the wires converged in one bundle with a bundle diameter of ˜26 μm (FIG. 1C). Each fiber terminated in uninsulated carbon, in a conical profile. The length of this cone was 89±17 (SD) μm. The diameter of the bundle, along the full length of the array, is smaller than single wires in most commercial arrays (33-50 microns for all Tucker Davis microwire arrays and 25-50 microns for all Microprobes microwire arrays).

Considering the time required to insulate the carbon, burn the fibers, and test impedance, array construction typically takes 3-4 hours for an experienced electrode builder. In a sample of 16 arrays constructed by an expert, an average of 92±8% (±indicates SD) of the wires were functional (n=210 fibers), where functional electrodes are defined as having an impedance lower than 4 MΩ. For an experienced electrode builder, the failure rate is low.

Surgical Procedure

All procedures were approved by the Institutional Animal Care and Use Committee of Boston University (protocol number 09-007). Zebra finches (n=14; 4 for acute experiments, 10 for chronic HVC recordings and 2 for intracellular-like recordings) (>120 days post-hatch) were anesthetized with 4.0% isoflurane and maintained at 1-2% isoflurane during the aseptic surgical procedure. The analgesic Meloxicam (120 μL) was injected intramuscularly into the breast at the start of the procedure and the animal was placed into a stereotaxic instrument. Feathers were removed from the scalp and a Betadyne solution applied. Bupivicane (50 μL) was then injected subcutaneously into the scalp before an incision was made along the AP axis.

The skull over HVC was localized using stereotactic coordinates (30° head angle; 0.7 mm AP, 2.3 mm ML, 0.4-0.7 mm DV), and the outer bone leaflet removed at the location of HVC with a dental drill. The lower bone leaflet was carefully removed with an ophthalmic scalpel, similar to implant procedures for recording with microdrives {Long:2010db}, exposing a hole of ˜100 μm diameter. A minimal durotomy was performed using an ophthalmic scalpel (typical durotomy was less than 50 microns.). Electrodes were mounted on a digital manipulator attached to the stereotax and slowly lowered through the durotomy. During insertion into the brain, the carbon fibers would occasionally begin to visibly splay. After lowering the array to the appropriate depth, the position in the song nucleus HVC was verified using antidromic stimulation from a bipolar electrode implanted in downstream Area X {Hahnloser:2002uv}. After verifying the position of the array, the craniotomy was covered with the silicone elastomer Kwik-Sil (World Precision Instruments) and the array was glued into place using light-bonded acrylic (Flow-It ALC, Pentron) along the entire length of the electrode shank, such that no portion of the carbon fiber bundle was left exposed or loose.

The same surgical procedure was followed for the acute recordings in Area X and HVC. For acute recordings in auditory area Field L, adult (>120 DPH) female zebra finches were anesthetized with 4% isoflurane (maintained at 1-2%) and a custom-made stainless steel head-post was glued to the scalp over the left hemisphere. The birds were then given several hours to recover, after which they were head-fixed in a soundproof chamber. Recordings were made from spontaneously active cells in Field L. Following the acute recordings, birds were euthanized using 200 μL sodium pentobarbital (250 mg/kg) injected into the breast muscle.

Electrophysiological Recording

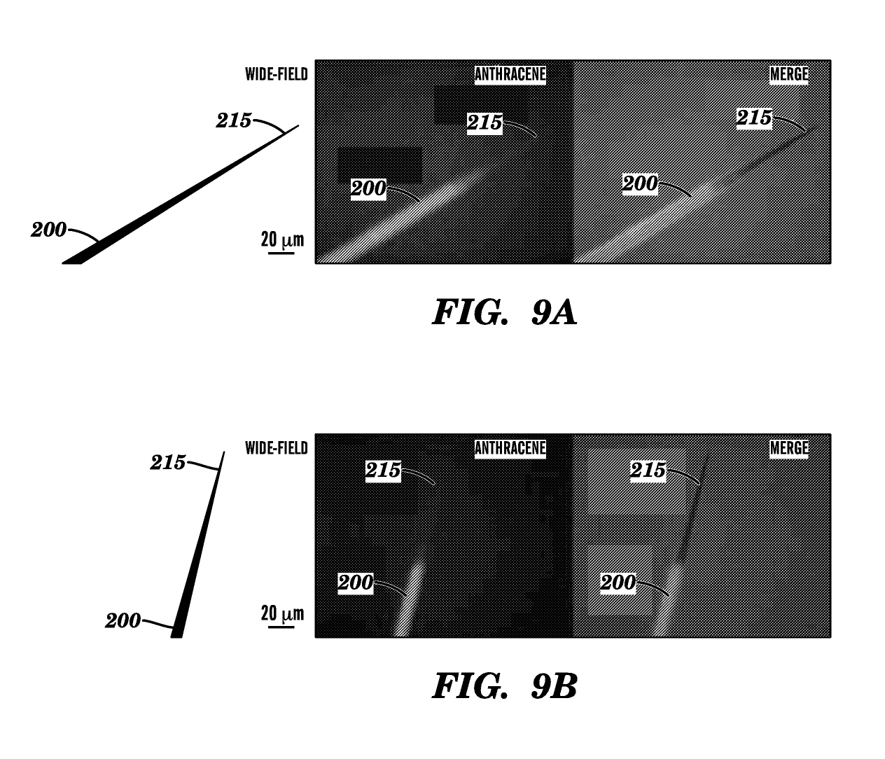

Acute recordings were performed using an RZ-5 BioAmp Processor and Medusa Pre-Amplifier (Tucker-Davis Technologies). Data was sampled at 25 kHz with filter cutoffs set to 300 Hz and 20 kHz. (Data from the acute recordings were not impacted by any aliasing due to the proximity of the high frequency filter and the sampling rate. See FIG. 9.)

FIG. 9A, left side, is a wide-field image of a carbon fiber coated in fluorescent Parylene-C, after it had been heat-sharpened. FIG. 9A, middle, is a UV filter image for the same fiber. FIG. 9A, right side, is a merged image of the wide-field and fluorescence view, which shows that the heat-sharpening method exposes an average of 89±17 microns. FIG. 9B includes images from another example fiber. The length of the exposed carbon tip was taken to be the distance from the point to the boundary of the full intensity Parylene edge.

To record from behaving birds, the present inventors used the Intan Technologies 16-channel multiplexing headstage (RHA2116 with unipolar inputs) paired with the RHA2000-EVAL board for acquisition at 25 kHz. These head stages were configured with a fixed 11 kHz lowpass filter. To send signals from the headstage to the evaluation board (RHA2000-EVAL), a custom flex PCB cable was designed that connected the headstage to a commutator (9-Channel SwivElectra, Crist Instruments), which then passed signal to the RHA2000-EVAL. All data was analyzed off-line using a series of custom MATLAB (Mathworks) scripts. During the experiment birds were recorded continuously for five days each week and left off the flex-PCB cable for two days a week. To record singing, a miniature microphone (Knowles Acoustics; Digi-Key catalog # EM-23046-P16) was glued to the Intan headstage and recorded through an electrode channel, following a protocol that is available online and provided in the attached Appendix. For the data recorded herein, the reference electrode for all channels and microphone was an uninsulated carbon fiber bundled along with 15 insulated fibers. Prior to spike sorting, this reference signal was occasionally replaced offline with a common average reference subtraction {Ludwig:2009ic}. In preliminary tests, referencing to one of the insulated electrodes also works well. In practice, the referencing is accomplished by bridging the reference channel on the Intan headstage to an arbitrary electrode pin on the Intan headstage. (Note that the online protocol for microphone recording referenced above should be modified to ground one of the electrodes, so that the reference signal can be recorded. Subtracting the reference from the microphone signal offline will provide a cleaner microphone signal, though in practice referencing the mic to the brain works well most of the time, since the two signal amplitudes are of very different scales.)

Data Analysis

Song bouts were detected by looking for threshold crossings in the average power of the microphone trace between 2-6 kHz. Song syllables were clustered using previously defined methods {Poole:2012im}. The present inventors used a manual cluster cut to train a support vector machine {Cortes:1995ie}, which subsequently identified all instances of that syllable automatically.

To analyze single-unit activity, voltage traces were aligned to singing and high pass filtered with an 800 Hz cutoff (2nd order Butterworth filter). A threshold was set to a 4σₙ, where σₙ is an estimate of the noise level,

$${σ}_{n} = median \{ \frac{\vert x \vert}{0.6745} \}$$

{Quiroga:2004jc}. After detecting positive- and negative-going threshold crossings, the present inventors took a 1.1 ms window centered on the threshold crossing and re-centered on the absolute minimum after interpolating by a factor of 8 using cubic splines. Features of the aligned spike windows were computed using principal components analysis {Abeles:1977ib}. The present inventors fit a mixture of a Gaussian model to the features using the Expectation Maximization algorithm {Dempster:1977ul}, and to detect the number of components in the mixture the present inventors fit models with 2-7 components, and chose the best model by minimum description length {Rissanen:1978ez}.

The present inventors assessed the quality of clustering using signal-to-noise ratio (SNR), defined as the peak-to-peak voltage of mean spike waveform divided by the six times the standard deviation of the signal with all spikes removed. Only units exceeding a SNR of 1.1 were included for additional analysis {Ludwig:2009ic}. Additionally, inter-spike-interval histograms (ISIHs) were checked for refractory period violations, and the L-ratio and Isolation Distance were used to assess the quality of unit isolation {SchmitzerTorbert:2005iu}. The spike-sorting analysis described above applies an accepted standard for “sorted” single units that results in signals of varying degrees of isolation.

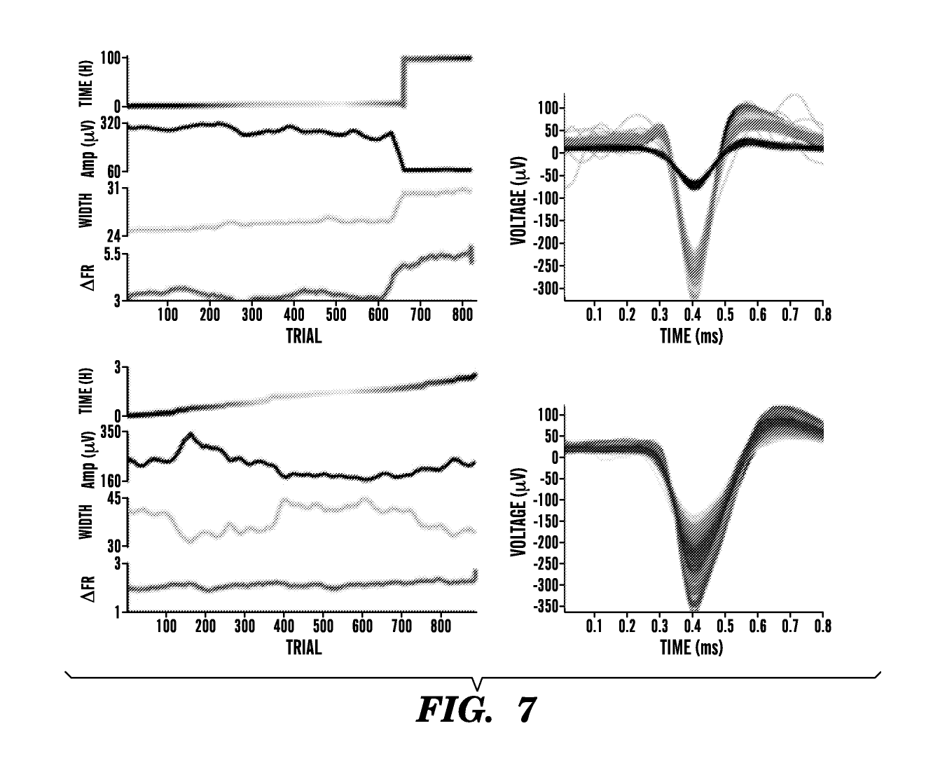

In addition to this analysis, the present inventors applied a more stringent analysis for unambiguous single units which required a minimum SNR of 2.8 and 0% ISI violations, which is sortable based on amplitude-threshold alone. For these “rigorous” single units, the present inventors also required stability of firing pattern as illustrated in FIG. 7.

FIG. 7 generally relates to example stability of spike features and firing pattern in rigorous single units. FIG. 7, left side, is a chart of the total elapsed time from the first trial (top), peak amplitude (second from top), spike width (second from bottom, in samples at 200 kHz) and root-mean-square error of the average instantaneous firing rate estimated in a sliding 25 trial window (bottom, see Methods) are shown across trials. FIG. 7, right side, is a trial-averaged spike waveform. In the top example, both spike features and the firing pattern sharply change on the same trial. The bottom example demonstrates the utility of a stable firing pattern. Though the spike features drift from trial to trial, the firing pattern remains stable, allowing for reliable unit identification through continuous changes in the waveform.

In what follows the present inventors report both on “standard single units”, or sorted multi-unit, and “rigorous single units,” making clear which criteria applies to each statement.

Sorted multi-unit and single-unit stability using spike features and spike train statistics

A critical question in chronic studies is whether two different clusters on separate days represent the same neuron. To approach this problem quantitatively, the present inventors used methods developed in {Tolias:2007dy, Dickey:2009wi, Fraser:2012bz}. In brief, the similarity between mean waveforms and inter-spike-interval histograms were used as measures of the likelihood that clusters across recordings sessions represent the sorted multi-unit ensemble site. For waveform similarity, the present inventors used the Fisher-transformed peak normalized cross-correlation; for ISIH similarity, the present inventors used the Jensen Shannon divergence {Lin:1991kv}, a commonly used probability distance measure.

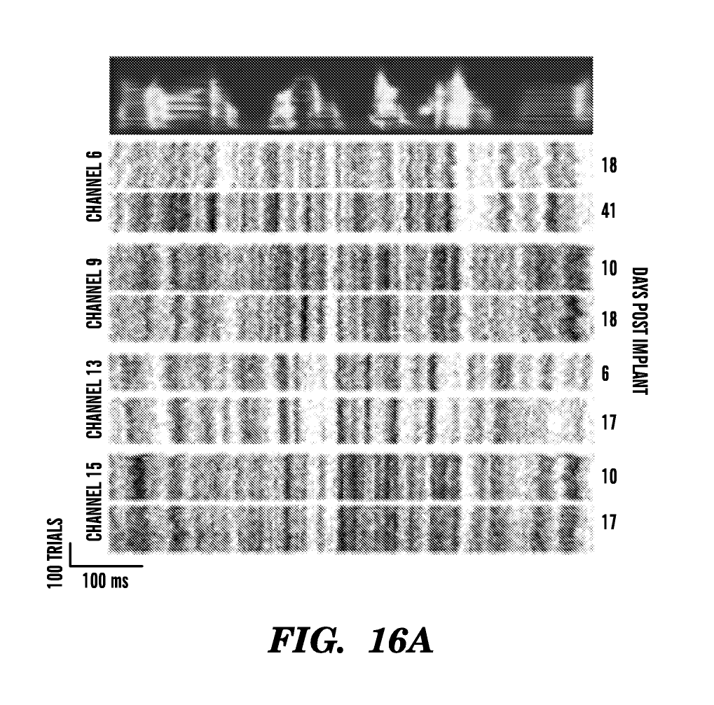

In HVC of an adult songbird, it is also possible to confirm recording stability by examining raster patterns across time. For neurons classified as stable by the methods described above, the present inventors found consistent firing patterns across time (FIG. 16). This point is discussed in greater detail in the results section.

To verify the stability of rigorous single-units, the present inventors continuously tracked the spike height, spike width, and change in firing pattern across bouts of singing. The change in firing pattern was assessed by computing the average smoothed instantaneous firing rate (Gaussian window, σ=5 ms) {Leonardo:2005 kw} over a 25 trial sliding window and taking the root-mean-square error between successive window averages. Synchronous changes in spike features and the firing pattern indicated that the single-unit signal had been lost or contaminated by other cells.

Imaging

Scanning Electron Microscopy images of the carbon fiber arrays were taken at the Boston University Photonics Center using a Zeiss Supra 55VP Field Emission Scanning Electron Microscope. Confocal images were taken with an Olympus 1X70 microscope. For fluorescence imaging of electrode tips, the present inventors coated UMS carbon fibers with Parylene-C containing 1% anthracene (141062 Sigma Aldrich) which fluoresces in ultraviolet light. Images were acquired using Olympus MagnaFIRE software and measurements of the length of the carbon fiber tip exposed from Parylene were made in Adobe Illustrator CS6 by merging the fluorescence with wide-field images.

Results

Heat-Sharpening the Array

The underwater firing process exposed 89±17 (SD)μm of insulation as measured by confocal microscopy (n=34 tips), leaving sharpened, uniform carbon fiber tips (see SEM images in FIG. 1C and fluorescent parylene images in FIG. 9). The torching process produced tips with an average impedance of 1.26 MΩ (n=210 tips) (FIG. 2A). Impedances measured in vivo after implanting showed a wide range of values in the weeks following implant (FIG. 2B).

FIG. 2 generally relates to electrode impedances. FIG. 2A is a histogram of the heat-sharpened pre-implant electrode impedance (n=210 fibers; median=1.0 MΩ). FIG. 2B is a chart of impedance of fibers in 7 implanted arrays measured at various time points after implanting. The pre-implant impedances (in saline) are shown at Day 0.

Acute Recordings

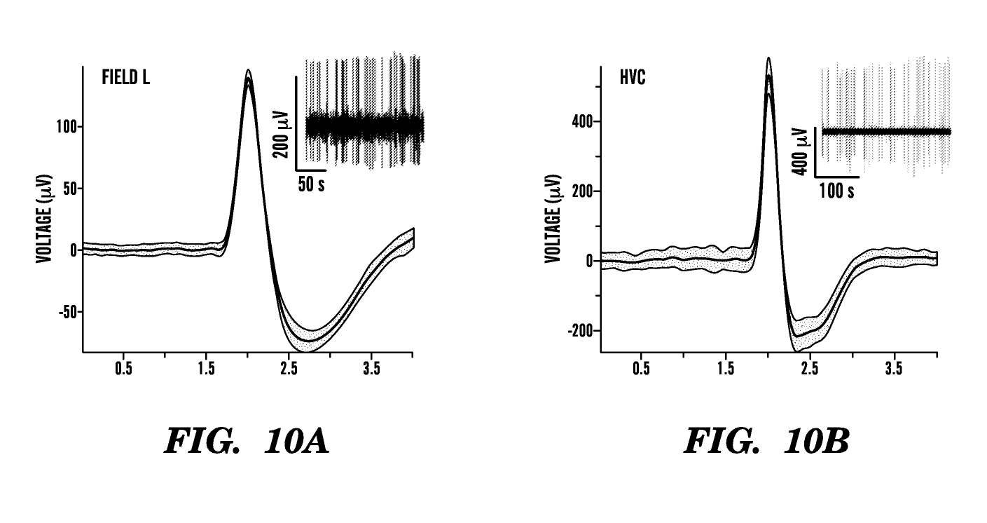

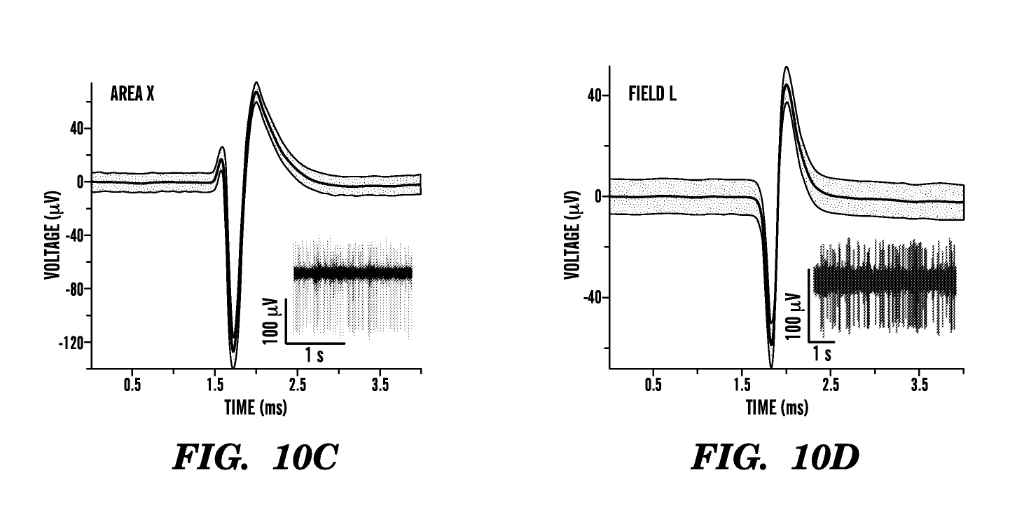

To initially assess the viability of carbon fiber electrode arrays for recording at various depths the present inventors recorded extracellular signal acutely in anesthetized or awake head-fixed birds (n=4 cells from 4 birds). FIG. 10 shows average waveforms from well-isolated neurons and spike trains recorded in auditory area Field L in awake head-fixed birds with an SNR of 9.18 and 3.00 (FIG. 10A and FIG. 10D, respectively); in premotor nucleus HVC in an anesthetized bird with an SNR of 21.64 (FIG. 10B) and in basal ganglia nucleus Area X in an anesthetized bird with an SNR of 3.50 (FIG. 10C). Carbon fiber arrays were thus able to measure signals from a range of cell types across a variety of brain regions, including a recording zone 3.0 mm deep (Area X).

FIG. 10 generally relates to average waveforms of isolated single units recorded acutely with 16-channel carbon fiber arrays. FIG. 10A includes results from a unit recorded in auditory area Field L in an awake head-fixed bird (SNR=9.18). FIG. 10B includes results from a unit from the pre-motor nucleus HVC in an anesthetized bird (SNR=21.64). FIG. 10C includes results from an isolated unit found in the basal ganglia of an anesthetized bird (SNR=3.50). FIG. 10D includes results from a unit recorded in Field L of an awake, head-fixed bird (SNR=3). Insets show corresponding spike trains. Inset scale bars are as follows: FIG. 10A: 200 μV, 50 s; FIG. 10B: 400 μV, 100 s; FIG. 10C: 100 μV, 1 s; and FIG. 10D: 100 μV, 5 s. The thickness of the line in the y-axis direction indicates the standard deviation.

Single-Unit Recordings in Freely Behaving Birds

A primary goal was to develop an electrode capable of tracking single units and small multi-unit clusters over extended periods of time. Thus, to assess the reliability and longevity of single cell signal, the present inventors implanted 16-channel carbon fiber arrays into the premotor nucleus HVC (n=10 birds), an area that produces precise neural firing patterns that ultimately drive muscular sequences to produce song {Long:2010db}. Spikes were aligned to all renditions of a given vocal element for a single day and displayed as a raster plot (FIG. 3).

FIG. 3 generally relates to a single unit recording in a singing bird and is an example of a putative interneuron recorded in the pre-motor nucleus HVC aligned to song. FIG. 3, top, is the time frequency histogram of aligned renditions of the same song motif. FIG. 3, middle and bottom, is a spike raster from a single unit aligned to song and a raw trace from the same channel, respectively.

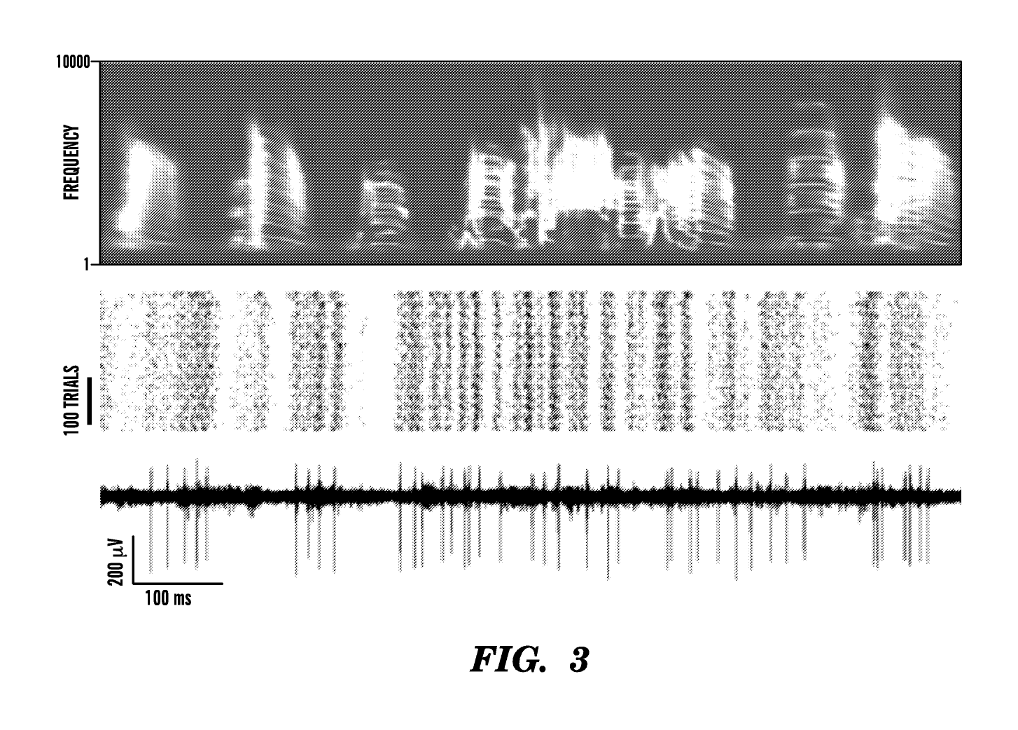

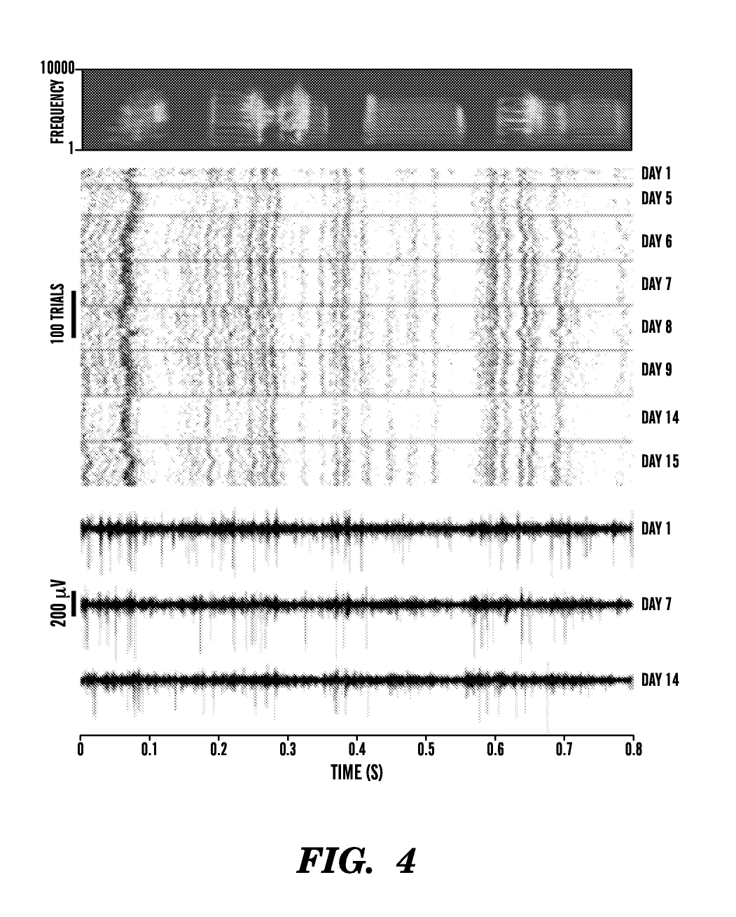

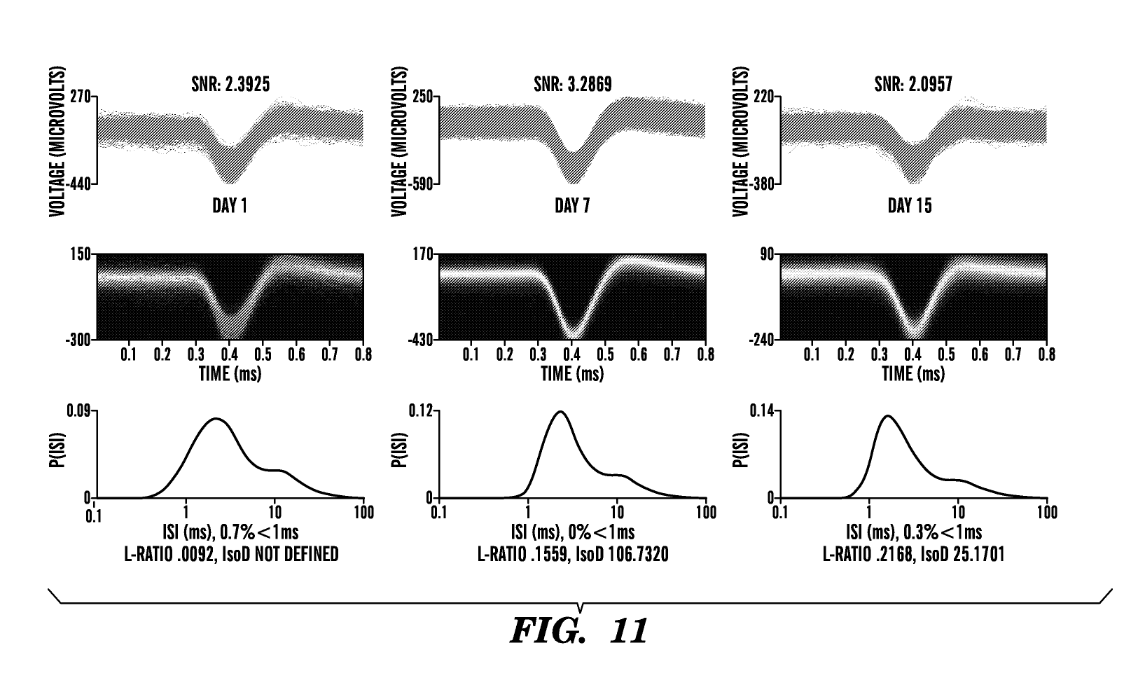

FIG. 4 shows one such raster from a putative HVC interneuron (classified as single-unit by the standard criterion—see Methods) recorded over a period of 15 days. Average waveforms and ISIHs from the 1st, 7th and 14th days are consistent throughout the period (FIG. 11). The average firing patterns are also stable over these time-scales.

FIG. 11 generally relates to clusters for the chronic signal shown in FIG. 3. FIG. 11, top row, shows overlaid spike waveforms. FIG. 11, middle row, shows spike waveform histograms. FIG. 11, bottom row, shows spike ISIHs. The SNR changed from 2.39 on Day 1 of recording (left column) to 3.29 on Day 7 (middle column) and 2.09 on Day 14 (right column).

FIG. 4 generally relates to chronic recording stability in the singing bird. FIG. 4, top, demonstrates a putative HVC interneuron (single unit by the standard criterion) in a bird recorded over 15 days. FIG. 4, bottom, shows raw traces from the same channel on Days 1, 7 and 14. Signal fading (as on day 14) indicates periods of partial loss of cell isolation.

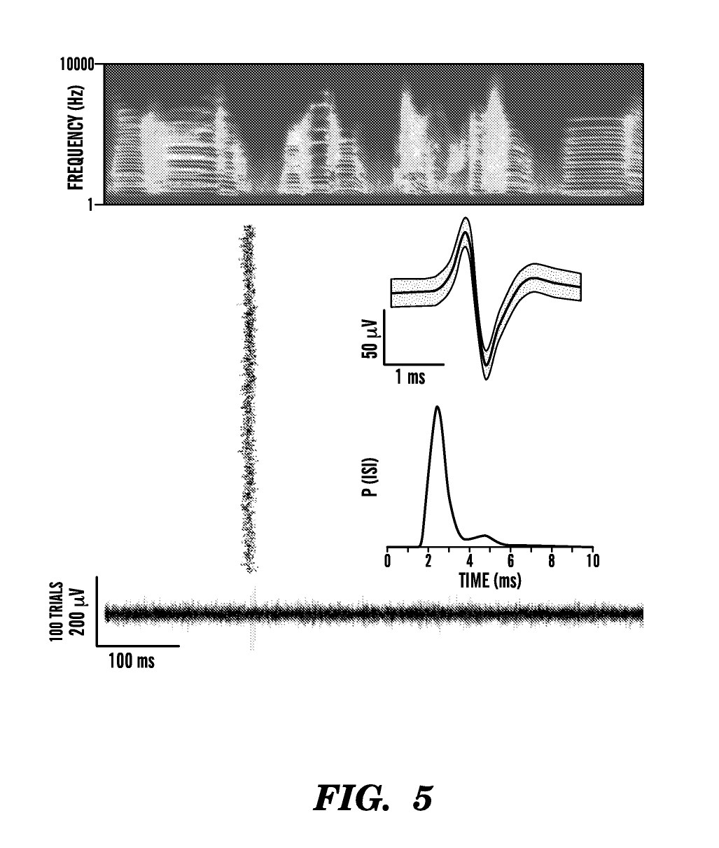

Additionally, in some cells the present inventors found distinctive waveforms and discharge patterns characteristic of principal neurons; that is, sparse high-frequency bursting aligned to a single point in the bird's song (FIG. 5).

FIG. 5 generally relates to a principal cell recorded in HVC. FIG. 5, top, shows a song-aligned spike raster of a putative RA-projecting neuron. FIG. 5, bottom, shows the raw voltage trace from rendition 199 out of 500 song renditions recorded across 1 hour and 26 minutes. Insets show the average waveform with SD and ISI distribution.

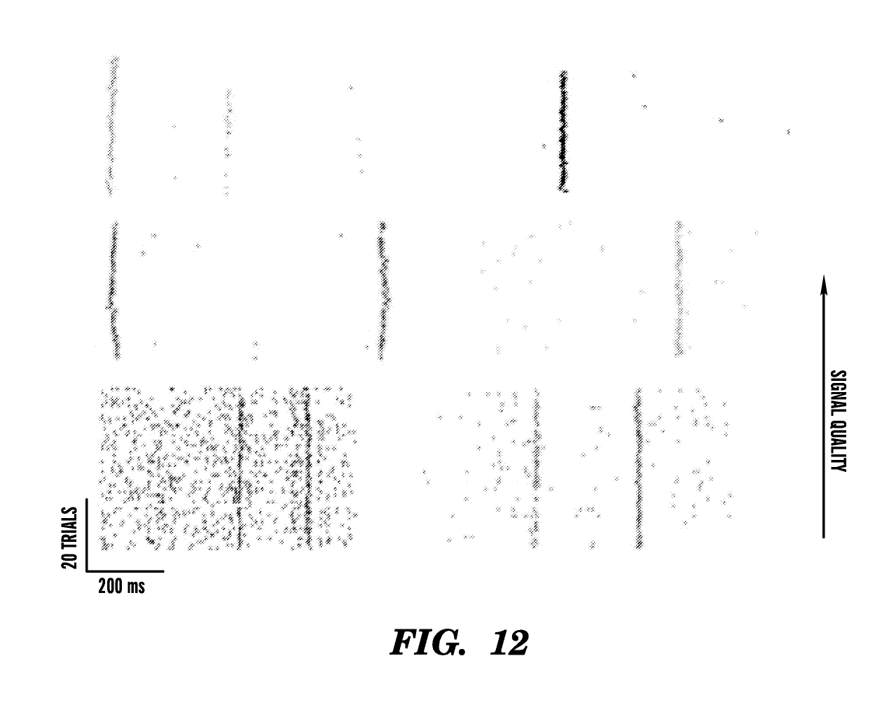

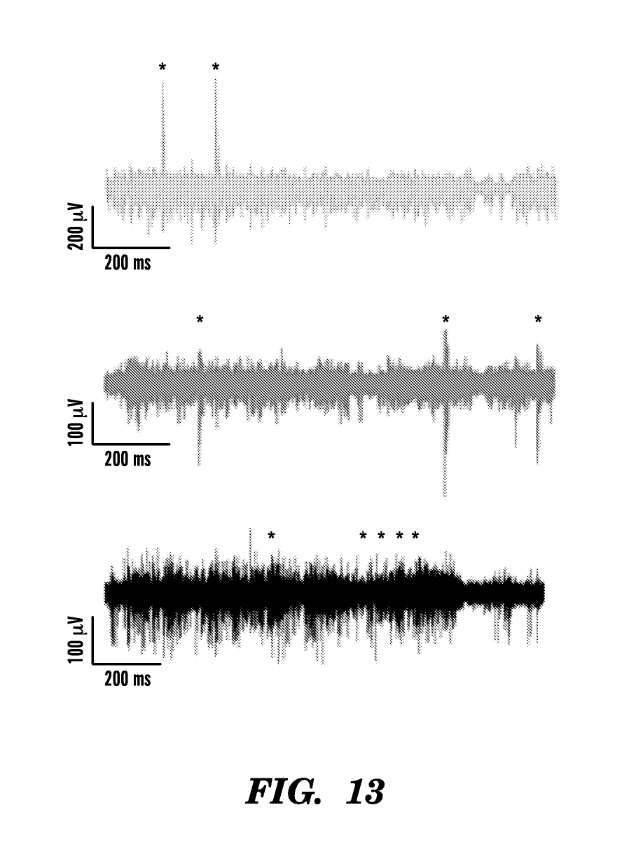

The cell recorded for this figure met the standard criterion for single unit isolation (projection neurons of various signal qualities are shown in FIG. 12 and FIG. 13).

FIG. 12 shows example projection neuron recordings of various signal qualities. Song-aligned rasters from 6 neurons show the effect of signal quality on unit isolation. As signal quality decreases, the number of contaminating non-burst “error” spikes increases. All units except the one shown in the bottom left raster meet the standard criteria for single units (see Methods). The raster in the top left met the more stringent criterion.

FIG. 13 shows single trial voltage traces from the 3 rasters shown in FIG. 12. The single trial voltage traces are shown with the identified spikes indicated by an asterisk.

Prior recordings of this neuron type have required high impedance electrodes mounted on motorized microdrives that allow for fine positioning of the electrode in the vicinity of the neuron {Hahnloser:2002uv}. This is the first report of HVC projection neuron recordings from immobile chronic implants. The yield and longevity of all recorded neuron types are reported below.

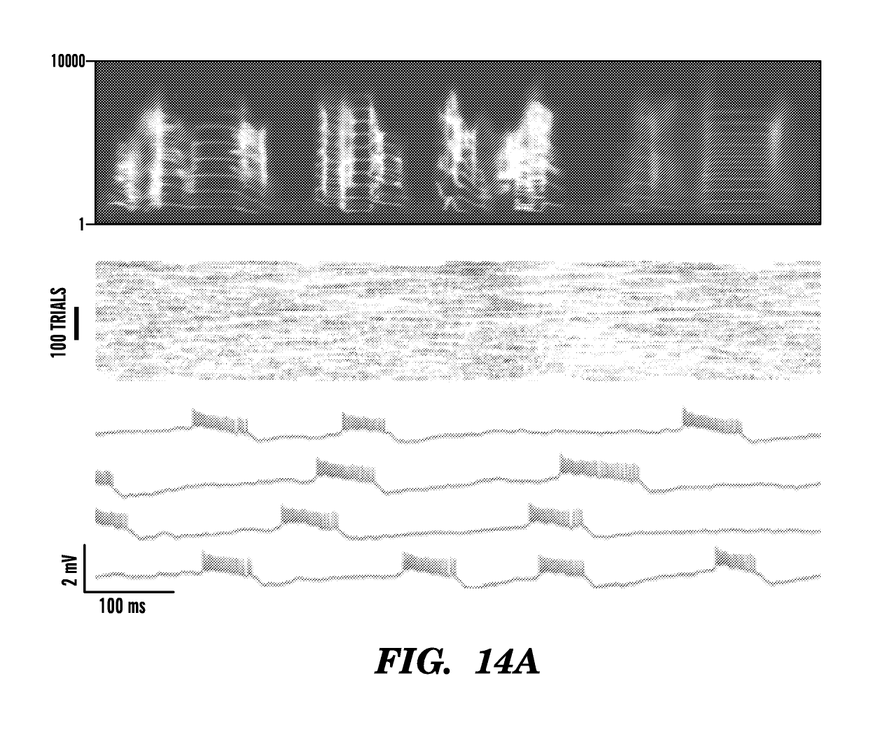

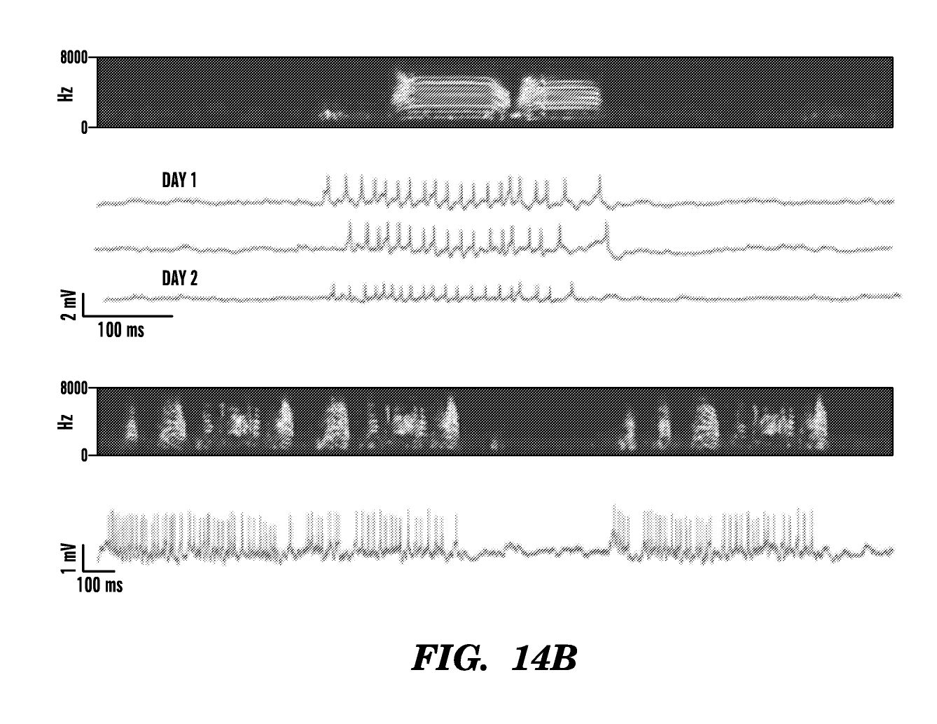

On two occasions, a portion of the sharpened tip of an electrode apparently entered a cell, yielding intracellular-like traces that were stably held for 12 hours in one case and 36 hours in the next (FIG. 14).

FIG. 14A shows a bursting cell with high amplitude positive peaks recorded from a bird implanted in Area X. FIG. 14A, top is a sonogram of the song; FIG. 14A, middle, is a spike raster; and FIG. 14A, bottom, shows low-pass filtered traces showing prominent LFP modulation during spiking FIG. 14B shows a similar cell recorded from a bird implanted in HVC and includes unfiltered traces during a call, across two days (FIG. 14B, top); and during singing (FIG. 14B, bottom).

The intracellular-like cells recorded in area X and HVC were characterized by high amplitude spikes and positive subthreshold voltage ramps prior to spikes or bursts and stereotyped hyperpolarizing potentials following the burst in the area X cell. In these rare recordings a portion of the uninsulated (80 micron) tip must have remained outside the cell {Angle:2012ii}.

Simultaneously Recorded Traces

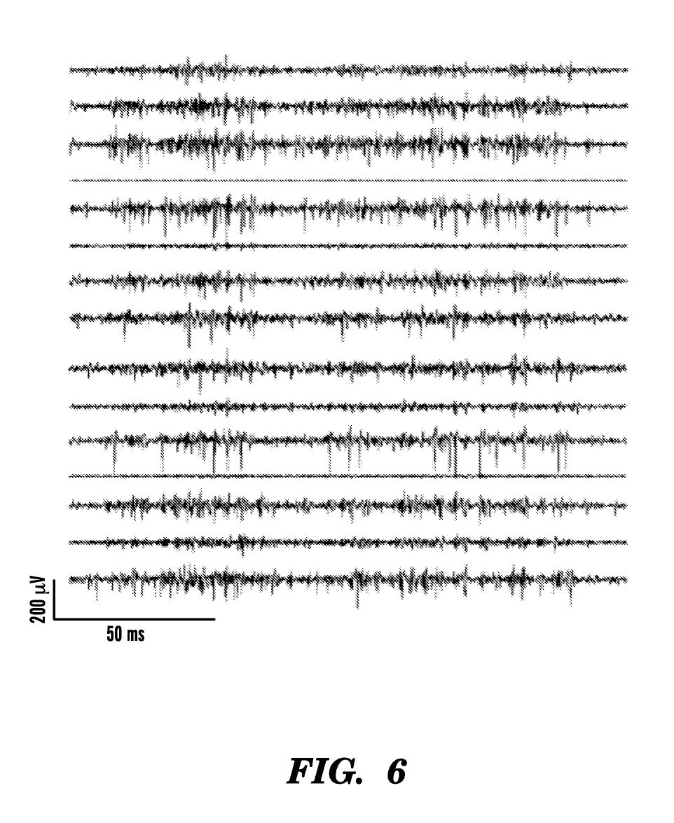

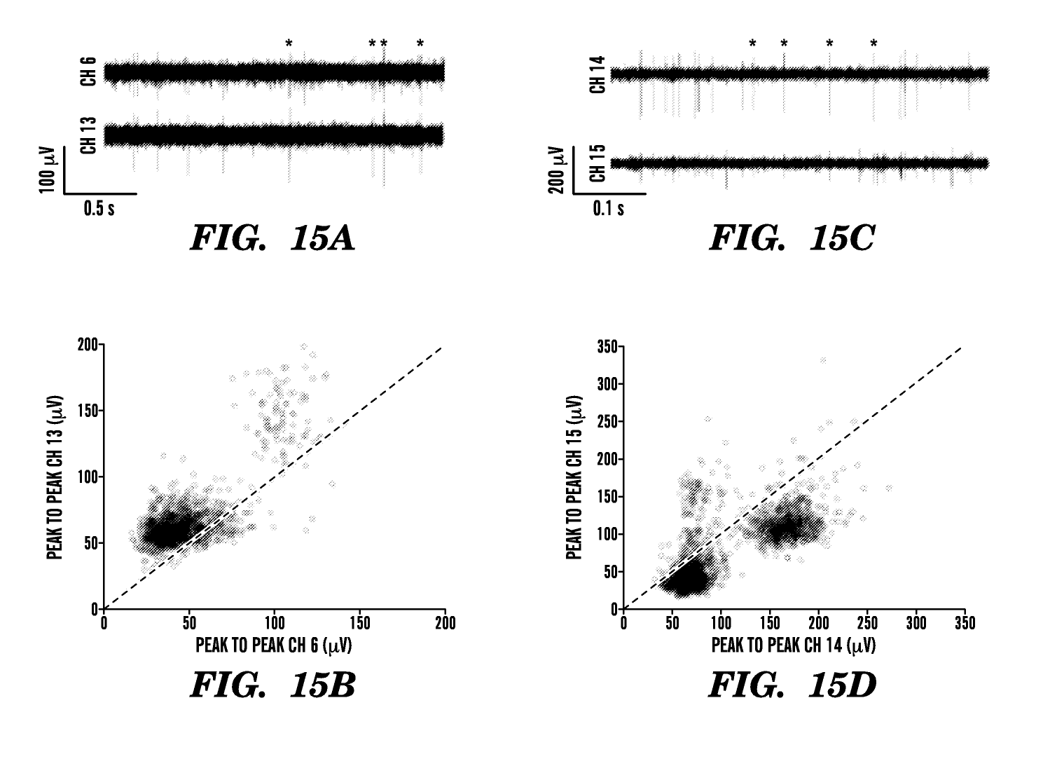

Of particular interest in multi-electrode recordings is not only the longevity of single-unit signal, but the ability to record multiple signals at once. In chronic implants of carbon fiber arrays, high quality multi-unit signal was often present on the majority of electrode contacts, though multiple “rigorous” single units were not recorded simultaneously on any implant. FIG. 6 shows fifteen simultaneously recorded multi-unit channels five days post-implant out of sixteen total channels (fourth channel from the top is bridged to reference; channel not shown is the microphone trace). With the small diameter and proximity of electrodes, individual neurons were occasionally visible on multiple channels simultaneously. Features of correlated signal across channels (i.e. tetrode effect) are commonly necessary to isolate densely packed neurons, but these features were not used here. However, FIG. 15 shows two examples of channels with common signal on two channels from birds implanted with 16-channel arrays. This figure illustrates the potential of improving single unit isolation based on multi-electrode features in future carbon fiber electrode designs.

FIG. 15 generally relates to the tetrode effect. FIG. 15A and FIG. 15C are example traces recorded from a chronically implanted bird showing correlated signal on two channels. Stars indicate example times for spikes present on both channels. FIG. 15B and FIG. 15D are scatter plots of spike amplitudes on the two channels showing correlated signal. Events do not fall on the unity line (dotted line), suggesting that the common signal is not explained by cross-talk.

Yield and Single Unit Stability

Single units defined by standard criteria were defined as containing (1) adequate SNR (>1.1) and (2) a minimal fraction of ISIs shorter than a refractory period of 1 ms (<5%). After discarding clustered units that did not meet these criteria, the present inventors recorded an average of 5.3 neurons per bird as defined by the standard criterion (see Methods) (n=6) that ranged from as few as 2 neurons to as many as 8. This count includes both putative interneurons and projection neurons, classified according to their firing patterns.

Four rigorous single units (n=3 interneurons and n=1 projection neuron) (SNR>2.8 and 0% ISI violations) were found in a set of n=4 implants. Finally, 16 projection neurons that did not meet the rigorous single unit criteria were recorded in at total of n=10 implants (SNR<2.8, though for this population all cells had 0% ISI violations except two, which had 0.3%). These cells produce a single burst one or two stereotyped times in the song, and single unit isolation could be confirmed in spite of the low SNR values. For this population of cells, the present inventors computed the fraction of contaminating spikes (spikes occurring within 100 ms of the burst, but outside a 10 ms window around the average burst time). The fraction of contaminating spikes ranged from F=0.425 in the most marginal case to F=0.008 in the best case, with an average of 0.128±0.135 (SD). Examples of these raster plots are illustrated in FIG. 12.

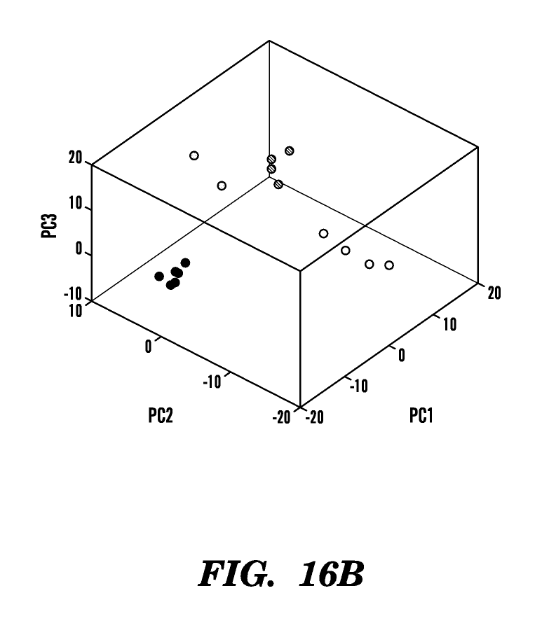

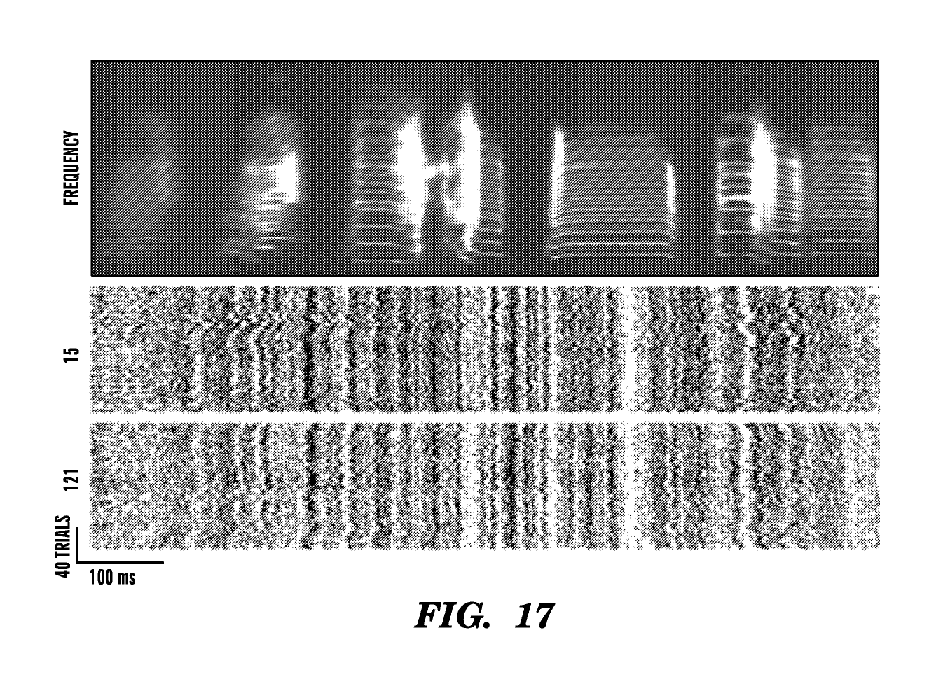

To assess the stability of isolated units, the present inventors followed the methodology of {Dickey:2009wi, Fraser:2012bz} (see Methods). From one day to the next, a stable waveform indicated continuity of recording from a single neuron. However, considering the population of all neurons recorded, waveform shapes were not unique, nor were ISIHs. One solution is to increase the dimensionality of the waveform shape by considering projections of the waveform on additional channels. However, in HVC of adult songbirds, a more powerful approach is possible based on the unique spike patterns produced by different neurons in HVC {Hahnloser:2002uv, Kozhevnikov:2007eu}. For each neuron observed here, spike timing patterns were unique and stable across time (FIG. 16 and FIG. 17.). This is true both for clusters consisting of small numbers of cells (FIG. 16), single interneurons (FIG. 7), and projection neurons (FIG. 12).

FIG. 16 generally relates to Sorted Multi-unit Stability. FIG. 16A shows four signals recorded in HVC on different channels in one bird. Each site was recorded on two sessions for each neuron. (Units were sortable by the standard criterion.) Firing patterns on different electrodes are distinct across the small ensembles, but similar for any given ensemble's two recording sessions. Days post implant of recordings are shown to the right of each raster. FIG. 16B shows principal components analysis of the firing rate patterns shown in FIG. 16A. Each dot indicates the average projection of the firing patterns for an entire day.

FIG. 17 shows an HVC interneuron recorded on sessions 107 days apart. Analysis on waveform shape, ISI distribution and firing pattern classifies the recordings as the same sorted cell, based on the standard criterion. Dates post implant are shown on the left of each raster.

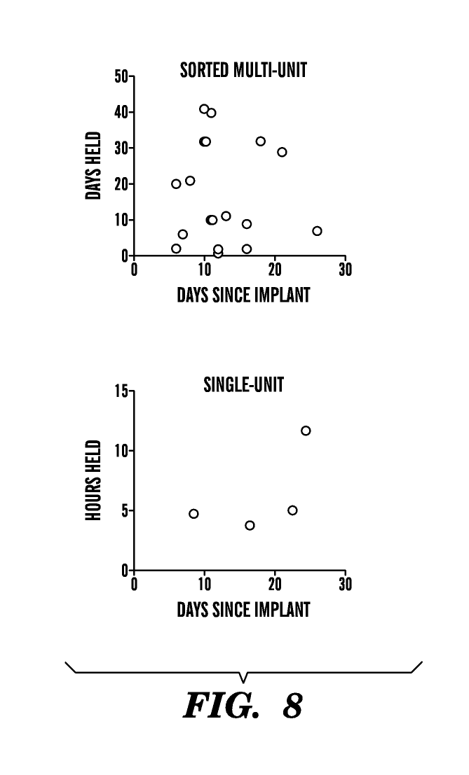

Of the 27 neurons that passed the standard SNR and ISIH quality criteria (n=6 implants), the present inventors analyzed the 18 standard quality cells that were stable for more than one day (for the other 9, no suitable clusters on the same electrode channel were found after the first recording session). Of this set of 18, the cluster quality varied, with 5/18 having <1% ISI violations and the rest <5%. The longevity of each recording is shown for each of 18 standard cells in FIG. 8. Of this total, 11/18 were stable for one week, 6/18 for two weeks, and 4/18 for 30 days. Projection neurons were not recorded for more than 2 days.

FIG. 8 generally shows stability of sorted multi-units and single-units. FIG. 8, top, charts stability of sorted multi-units. These points represent single units by standard criteria. The 107 day example from FIG. 17 is excluded from this plot. FIG. 8, bottom, charts stability of rigorous single-units, which were isolatable based on threshold alone.

For the rigorous single units (n=4) the present inventors analyzed all putative cell types. The longevity of each single unit recording is shown for each of 4 neurons in FIG. 8, which ranges from 4 to 12 hours. (Outside of this time-scale, the cells fell below the threshold for rigorous single units, though they were still isolatable based on standard criteria that allowed for some error.)

### DISCUSSION

The carbon “microthread” electrode array provides a stable interface to record small neurons in singing birds. The present inventors have shown that the arrays yield stable signals over time-scales of weeks, with occasional examples over time-scales of months (FIG. 17). The process of unit isolation described here did not take advantage of the occasional appearance of neurons on multiple channels of the electrode bundle; exploiting multi-channel features (through the tetrode effect, FIG. 15) would likely increase the yield and stability of single units recording with this array. Over the time-scale of the recordings, individually unique firing patterns in HVC were stable, allowing confirmation of the independent measures of stability based on waveform and ISI distribution. For the zebra finch, ground truth for neural stability is available; distinctive firing patterns provided the added information needed to confirm recording stability in an automated analysis. This approach can be compared to the utility of studying neural interfaces in areas with distinct sensory, motor, or place-field responses that can aid in single neuron identification.

The carbon fiber electrodes differ from commercially available arrays in a number of respects—and in particular the small scale. The constraint of planar wiring patterns in silicon arrays leads to relatively wide shanks in most electrode geometries (minimum 47 μm shank width (e.g., the Neuronexus Buzsaki64sp probe; 125 μm shank spacing; 15 μm thickness). In other silicon arrays, the electrode tips are small, but the taper on each electrode expands rapidly (e.g., electrodes are 80 μm in diameter at the base and taper down to a point—{Jones:1992vq}). This large diameter can lead to significant tissue damage and gliosis {Polikov:2005cq, Biran:2005dm}. One embodiment of the carbon fiber bundle of the present invention is comparable in diameter (26 microns) to many single microwires (12-50 microns {Gray:1995fg, Nicolelis:2008vl}), and the thin profile holds not only for the tip, but along the full length of the electrode. The process of implanting the electrode is, as a result, minimally invasive {Kozai:amAEGRtY}. The dense tip geometry and thin shank can be particularly useful for targeting deep brain structures. Carbon fiber is available in a range of stiffnesses, and the relative ease of implanting the carbon bundle suggests that even more flexible fibers can be implantable in the same geometry, particularly if they are first stiffened by a dissolvable substrate {Chorover:1972cc, Kim:2010kk}.

With practice, assembly times for a 16 channel array are 3-4 hours, per array, including all steps from carbon insulation through tip preparation. Methods that can accelerate this time are anticipated. If the small geometry or other material properties of the carbon bundle can be definitively associated with increased stability of neural recordings, then a search for manufacturing processes that can scale up the number of contacts or efficiency of construction is well-motivated.

The carbon bundles reported here provide the first long-term recordings in nucleus HVC of singing birds, and the first report of projection neurons in HVC isolated with immobile implants. The chronic stability of the carbon fiber signal is striking, but the biggest limitation in the present data set is the scarcity of high SNR single unit recordings that allow for unambiguous isolation of single cells based on spike threshold alone. While cells of this “rigorous single unit” quality did appear in the data set, they were rare, and most of the data reported here is “single unit” by standard spike clustering measures that allow for a significant degree of mislabeled spikes. The recording tip of the electrode is 80 microns long, and given the length scale of this uninsulated tip, it is surprising that single unit isolation in HVC is possible in singing birds. In HVC, 10-15 micron diameter projection neurons fire in a background of highly synchronous interneuronal activity, making isolation of this cell type extremely challenging. The path forward for this design will require improved control of the electrode bundle geometry so that multi-channel features can be used more frequently for single cell isolation. In parallel, smaller tip geometries could improve isolation, though maintaining low impedance for smaller tip geometries will require modifications of the recording surface through conductive polymers {Kozai:2012 bp}, attachment of carbon nanotubes {Keefer:2008ep}, or other surface modifications. Systematic comparisons of this design with other fixed electrode technologies are also needed in preparations such as rodent hippocampus where background data is available for other electrode types. With the current carbon fiber electrode bundle, firing patterns of small clusters of cells in HVC can be tracked over time-scales of learning in songbirds. Occasionally, rigorous single units can be isolated over time-scales of hours to days. Carbon fiber electrodes may provide a scalable solution for chronic recording of densely packed cells in small animals.

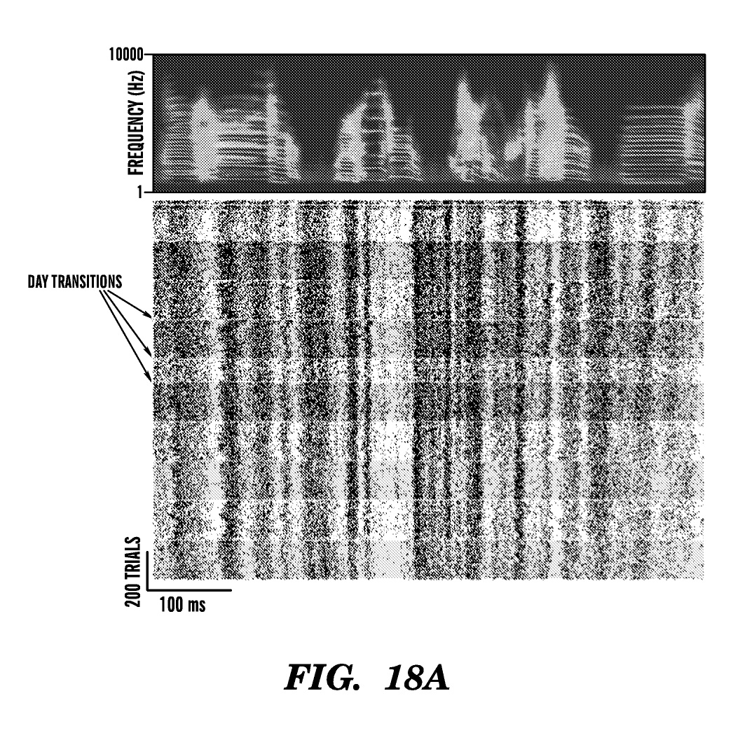

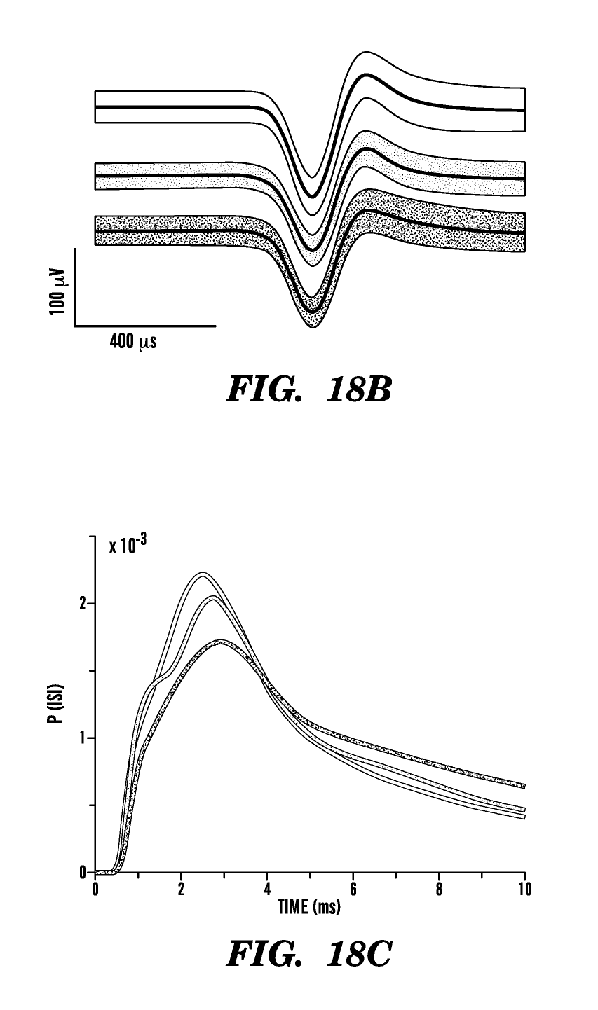

FIG. 18 generally relates to single unit stability; FIG. 18A depicts a putative HVC interneuron recorded on twelve sessions across 23 days; FIG. 18B is a chart of Average (+SD) waveforms on the first (top), sixth (middle) and last (bottom) days; and FIG. 18C is a chart of corresponding ISI distributions.

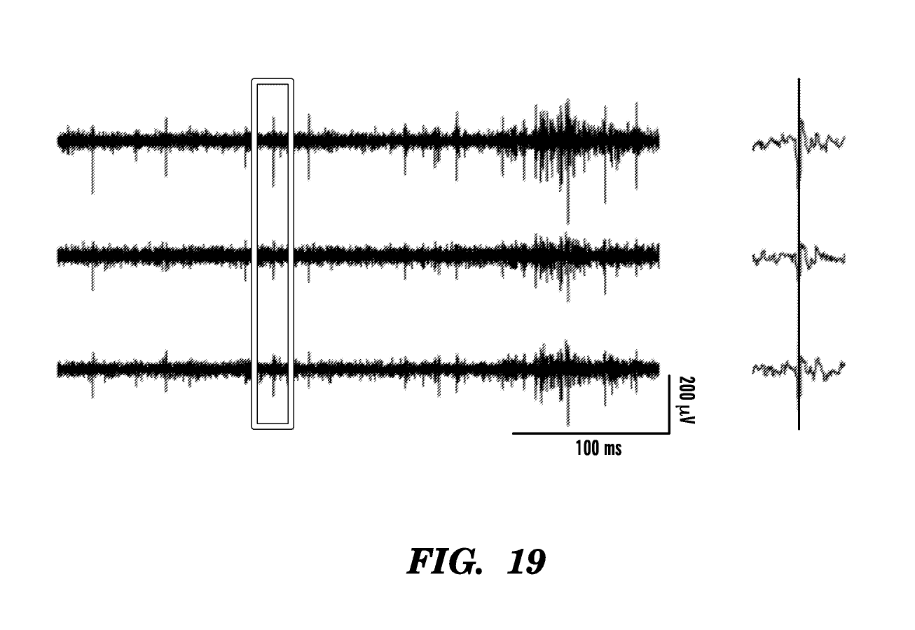

FIG. 19 includes the results of three channels recorded from a carbon tetrode.

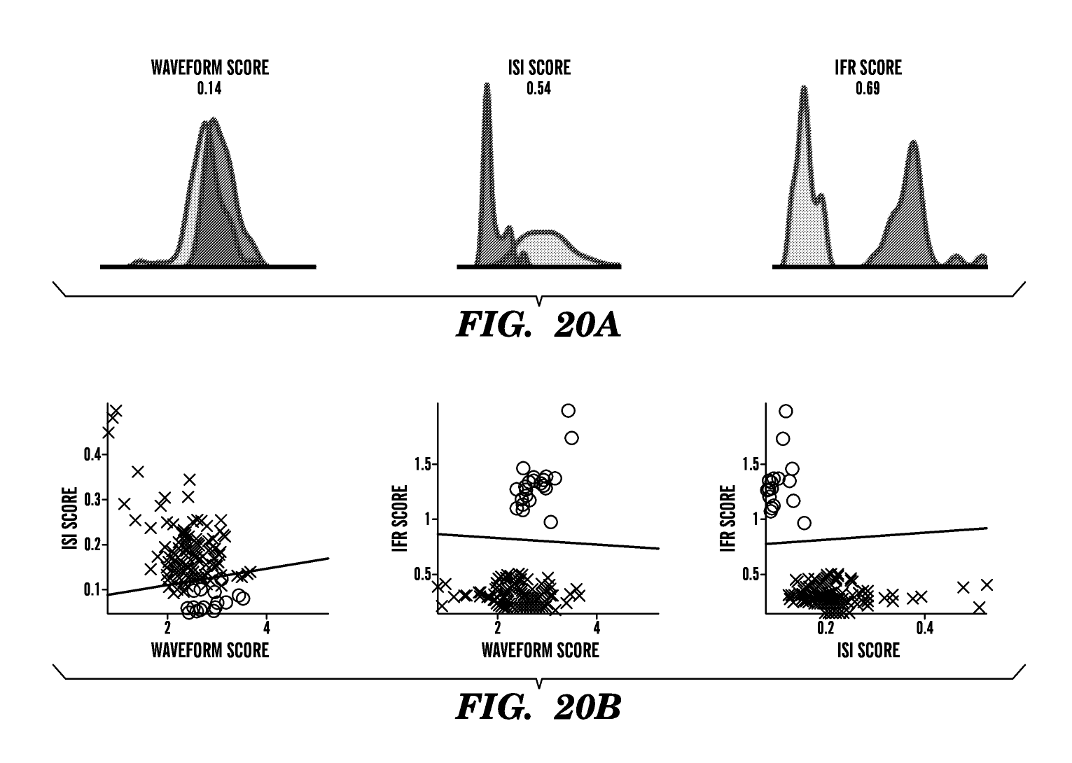

FIG. 20A depicts distributions for waveform, ISI, and IFR scores for stable single units (black) and the full ensemble of units recorded (gray), quantified with Jensen-Shannon Divergence; FIG. 20B depicts decision boundaries drawn using the three measures; and FIG. 20C is a beeswarm plot of the longevity of neurons held for more than a single recording session (18/27 interneurons) according to a classifier according to the present invention. The present inventors implanted 6 birds and recorded an average of 5.3 interneurons per bird (fixed implants). In addition 5 projection neurons were recorded that did not enter this stability analysis.

Research Strategy

Significance

Chronically implanted microelectrodes are central to scientific studies of neural circuit function in behaving animals. The technology has also been used to create intracranial brain-machine interfaces in humans[1] that restore communication or movement to locked-in, or tetraplegic patients[1][2][3][4][5], and the potential value of a precise, stable neural interface for the large population of amputees is clear. For basic research, visionaries foresee a time in the near future when researchers will be able to peer into the brain with vast numbers of probes that sense neural activity, and in the words of the Obama Brain Research through Advancing Innovative Neurotechnologies (BRAIN) initiative, “revolutionize our understanding of the human mind”[6][7][8]. However, both optical and electrical methods of neural activity monitoring face challenging technological limitations.

For microelectrodes, the biggest limitation of chronic recording is a reactive tissue response that encapsulates the electrodes and kills or damages neurons[9]. This limitation is particularly acute for basic research studies involving long term recording from the brains of small animals such as mice and songbirds. For a commercial silicon array whose effective feature diameter is typically 100 μm, histological markers of gliosis and neuron death reveal tissue damage extending up to 300 μm or more from the implant[10]. This length-scale of tissue damage does not prohibit occasional long term recording[4] from pyramidal neurons in primate cortex whose large polarized dendrites and large somas (up to 100 μm) produce a strong signal for extracellular recording. However, the length scale of tissue damage becomes prohibitive when recording from most cell types in smaller organisms. Examples of multi-day recordings from single cells in mouse hippocampus can be found[11], and a few notable chronic brain computer interface experiments have been performed in rodents[2][12], but recording methods that improve the efficiency of stable recording are needed. One goal in electrode design is clearly to minimize any damage to surrounding neurons and tissue.

The present invention develops a minimally invasive electrode array based on a new principle of “splayable electrode threads.” For example, an electrode according to the present invention can be bundled on a surface of biological material, such as brain tissue of a subject. The electrode can splay during and/or after implantation into the brain tissue. The splaying of the electrode can be a material property of the electrode. This feature can be important because a bundled fiber is easier to implant and less destructive to the surrounding brain tissue. The splaying action of the present electrode is desirable during and/or after implantation to allow electrode tips and does not cause significant destruction to the surrounding brain tissue.

The fibers can be together in a bundle on the surface of the implant target (for example, brain tissue) and can splay in an expanding cone as one looks deeper into the target (for example, as one looks deeper into the brain of the subject). This splaying does not necessarily occur over a significant amount of time, but can occur immediately during implant. The electrode fibers can be adapted so as not to be forced to splay. The electrode fibers can be adapted to have a material property of an initial bias towards bundling, so that the electrode fibers remain together, but can be adapted so that when the electrode is implanted, the fibers can be diverted by blood vessels and end up following diverging paths or “splaying” into the brain. The process can thus be characterized as a compliant splaying process, not a forcible splaying process.

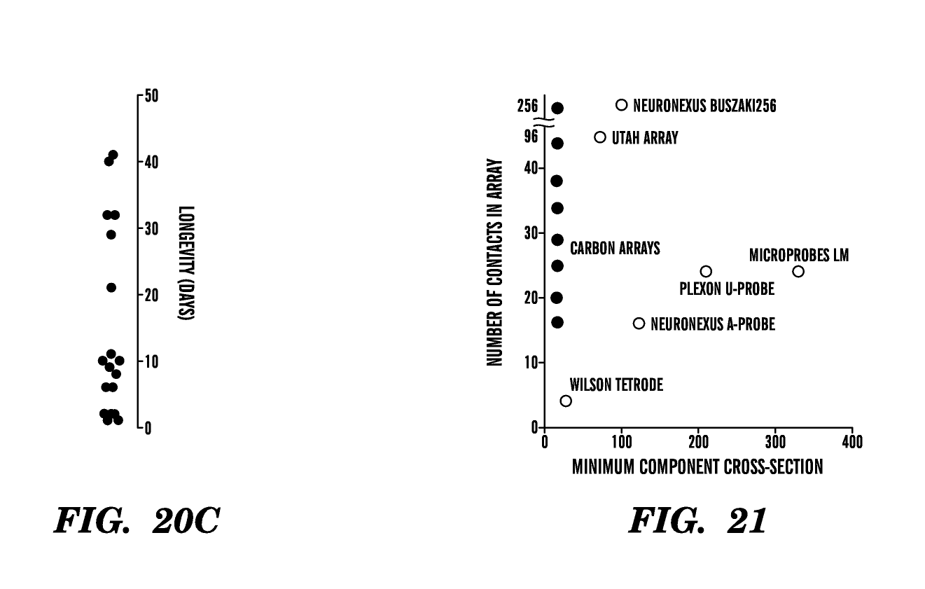

One goal is long term recordings in small animals. The proposed array occupies a region of electrode configuration space that was previously unoccupied (FIG. 21)—namely large channel count with sub-cellular (about 5 μm) individual shank size. By traditional construction methods, an array with features this small would be impossible to implant, since the individual threads buckle during the implant process. To produce an array with sub-cellular feature sizes, the proposed implant is held together at the surface of the brain by mutual attraction between the fibers, but mechanically separates during insertion, allowing individual threads to follow their own paths of least resistance into the brain. The present inventors hypothesize that this “tunneling electrode array” is minimally invasive, leading to a long term stable interface with neurons. For reasons discussed below, the present inventors benchmark this array in songbirds where a unique cross validation of spike stability is possible. The birdsong system embodies both the technical challenges of chronic recording in small animals (small, tightly packed neurons that are synchronously active), and the potential rewards of long term recordings that can track learning with single cell resolution. The present inventors anticipate that stable recordings will generalize from birds to other organisms and brain areas, making a range of basic research questions more tractable, such as tracking firing patterns of single neurons in small animals through learning. For example, tests in rodents are now underway, and the goal of seeing this method refined in other labs is supported by a data sharing plan that will provide electrode designs and expertise on a public web page and FAQ. In parallel to the present invention, efforts to address chemical surface treatments of electrodes that reduce tissue inflammation are promising[13]. The present inventors anticipate that tunneling fiber arrays, combined with other approaches will provide a qualitative leap in brain computer interfaces for research and for human prosthetics in the decade to come. To that end, the present inventors have the support of leading electrode development groups interested in combining their complementary approaches with the proposed design in future tests. The small animal model is a challenging test bed for chronic recording, and for this reason, gains in single unit stability in small animals are likely to generalize to animals with larger, less densely packed neurons, and eventually to human neuroprosthetic applications.

Innovation

Increasing attention to the limitations of current microelectrode technologies has indicated that the cross-section and stiffness of implanted electrodes must be minimized to reduce chronic disruption of the blood brain barrier[10][14][15]. A number of methods have been proposed for minimally invasive electrodes including wire bundles whose structural support dissolves upon implantation[16][17][18][19], or electrodes composed of more flexible polymer wires[20][21]. Sub-cellular scale carbon fiber “ultramicroelectrodes” have recently been proposed to reduce damage upon implantation relative to larger fibers and they also provide a reduced stiffness due to a parylene insulation layer[22] that is more compliant than glass[23]. While a number of minimally invasive single electrodes have been proposed, the challenge of building a physically implantable multichannel array from sub-cellular scale fibers was previously unsolved. The present inventors presently describe a solution to this mechanical problem that provides a scalable electrode design.

Tunneling Microfiber Array Design

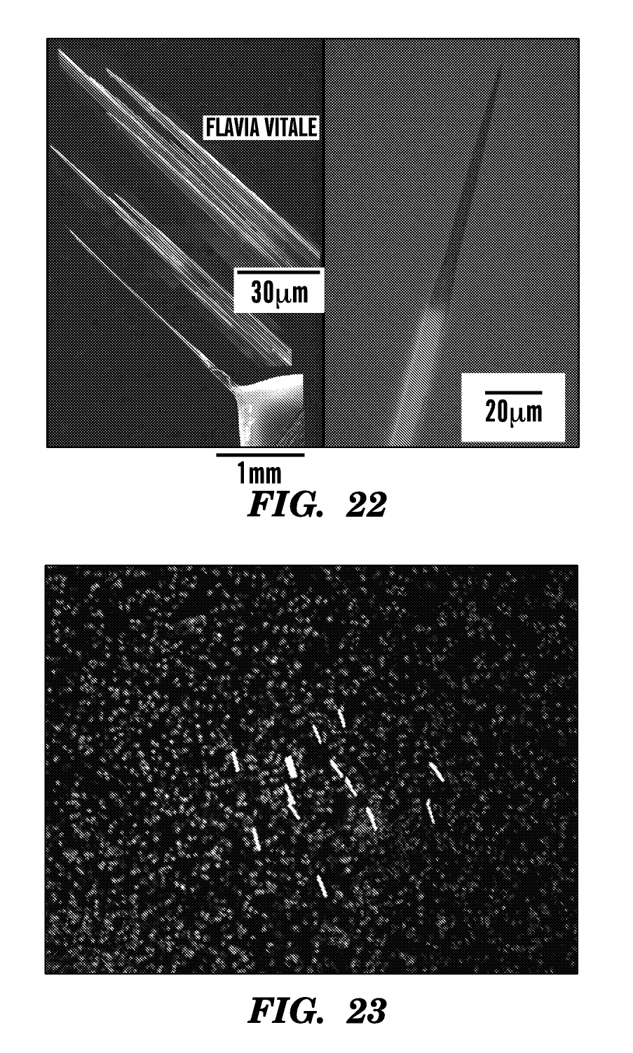

To create an implantable electrode array with sub-cellular scale fibers, parylene insulated carbon fibers (4 μm—FIG. 22) are threaded through a multi-channel funnel that is 3-D printed through stereolithography (FIG. 1A, FIG. 1B1, FIG. 1B2). Their top ends are connectorized in a single step by filling wells with silver paint and mating a connector to the electrode block. To produce a consistent low-impedance tip, the present inventors use either electrochemical polymerization of poly(3,4-ethylenedioxytheiophene) (PEDOT)[24] on blunt-cut carbon fibers, or a novel process that the present inventors developed for heat-sharpening with a gas/oxygen torch that exposes a larger surface area of the electrode. Heat-sharpening involves holding the array underwater while heating exposed tips (FIG. 1B1, FIG. 1B2). The water acts as a flame retardant or heat insulator, providing control over the amount of Parylene-C taken off of the tip of the carbon. In addition to sharpening the tips to sub micron diameters which facilitates implant, this removes a consistent quantity of insulation in the length of 89±17 (SD) μm, reducing the tip-impedance to an acceptable range for extracellular recording, i.e., 1.2 MΩ±300 kΩ (N=210).

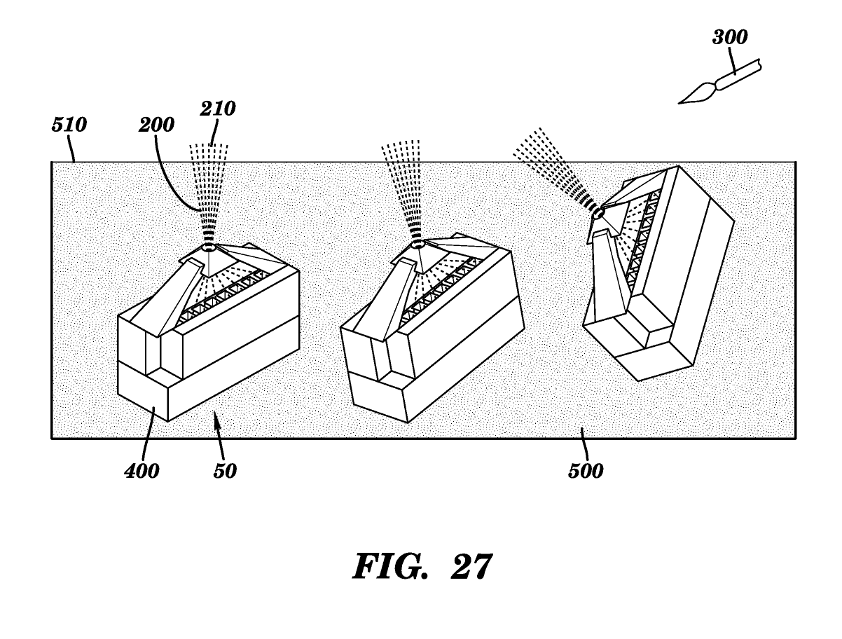

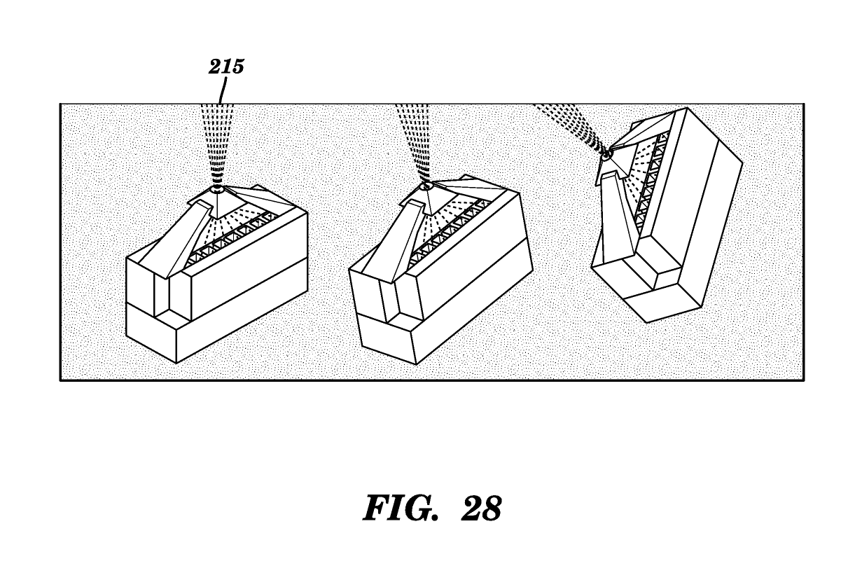

In a final step, the array is slowly drawn out of a water bath with the fibers directed in a substantially downward facing direction such that the fibers form a bundle. In this step, surface tension pulls together the fibers into a single bundle, and this bundle remains together after the fibers dry (FIG. 1B1, right). (After drying, the array is presumably held together by van der Waals force.) In the final construction, the fibers converge in one bundle with a diameter of ˜26 μm for a 16 channel device (FIG. 22), ˜36 μm for a 32 channel device, or ˜50 μm for a 64 channel device. Depending on the angle at which the bundle is held during heat-sharpening, electrodes can be built with tips of uniform length or a sloping profile for simultaneous recording at multiple depths. In sum, the electrode array is essentially self-assembled through the use of heat (such as flame) and surface tension-driven bundling at an air-liquid interface. In principle, this self-assembly can be scaled to arrays consisting of hundreds of contacts. FIG. 27 illustrates an example of an assembled array 50 lowered into a water bath 500 with the tips 210 of the carbon fibers 200 protruding above the surface 510 of the water 500, and the subsequent rotation of the array 50 at different angles prior to heating the tips of the fibers 200 by passing them through a heating source 300 such as a gas/oxygen torch. The angle is not limited to that illustrated in FIG. 27 and may be any suitable angle. FIG. 28 illustrates the array after heating the tips of the fibers 200 by passing them through a heating source 300 such as a gas/oxygen torch.

The previous paragraph emphasizes the simplicity of the assembly process, but the main value in the design is in the dynamics of the implant process. On the surface of the brain, individual fibers mutually support each other, and the bundled array can be easily implanted without buckling. However, once the array enters the brain, fibers gradually splay apart as the sharpened electrode tips pierce through the tissue (FIG. 23). The present inventors hypothesize that each fiber follows an individual path of least resistance in a compliant “tunneling process.” As such the array preserves the minimally invasive nature of the sub-cellular diameter fibers that compose the bundle.

Approach

One goal of the present invention is to develop and benchmark an electrode array capable of tracking single units and small multi-unit clusters over extended periods of time, in small animals. Quantifying the longevity of single unit analysis is typically challenging since spike waveforms are not unique, and waveforms drift over the course of a recording. For this reason, the present inventors chose to benchmark these electrodes in songbird cortical motor nucleus HVC (used here as a proper name). In HVC, unique and stable spike patterns produced across trials by each neuron type during singing allow a detailed quantitative analysis of spike stability[25][26][27] (FIG. 3, FIG. 4, FIG. 5, FIG. 16A, FIG. 17). In HVC, the firing pattern of a cell can be used in conjunction with spike waveform to cross-validate measures of recording stability since simultaneous shifts in spike waveform and spike firing pattern signal the loss of continuity in signal from one neuron (FIG. 7 and see above[27]).

To test the tunneling fiber array, the present inventors implanted 12 chronic implants with bundles of 16 fibers in each implant[27]. Each song was recorded from a head-mounted microphone in parallel with chronic neural data. Songs were acoustically aligned using methods the present inventors have reported previously[28][29]. Spikes corresponding to song are then displayed as a raster plot (FIG. 3, FIG. 4, FIG. 5, FIG. 16A, FIG. 17). Each recorded unit was found to produce a distinct stereotyped firing pattern, consistent with previous reports. Of the 27 neurons that passed standard SNR and ISIH quality criteria following the methodology of [3][30] (n=6 implants), the present inventors analyzed the 18 cells that were stable for more than one day. Of this set of 18, the cluster quality varied, with 5/18 having <1% ISI violations and the rest <5%. Of this total, 11/18 were stable for one week, 6/18 for two weeks, and 4/18 for 30 days or more (FIG. 1A, FIG. 1B1, FIG. 1B2). In HVC, somas are only 8-15 μm in diameter[31] and closely-packed in clusters making soma-soma contact[32]. Dendrites in HVC are also compact (40-100 μm radius), and spherical in shape rather than polarized[31][33][34], making this a challenging area to achieve neural recordings with single cell resolution. In prior studies it has not been possible to isolate single units in HVC with electrodes that were chronically fixed in a single location. The tunneling fiber array isolated single cells for days, and small multiunit clusters for months. The performance motivates a closer investigation of single unit yield and longevity, and the interaction with local tissue both in short term and long term time-scales.

Aim 1. Quantify the impact of fiber splaying on the yield and stability of neural recordings.

A useful feature of the proposed array is the property of electrode splay. This aim seeks to specifically document the relationship between electrode splay and signal yield and longevity by comparing the proposed electrode to a monolithic electrode whose fibers cannot splay. The latter electrode is prepared with an extra parylene deposition that ensheathes the entire bundle prior to tip sharpening. The monolithic electrode is 2 μm larger in overall cross section, and the fibers remain together during and after implant. The design has been confirmed in pilot tests (data not shown).

The present inventors will examine the isolation rate and stability of multi-unit and single unit recordings in cortical areas HVC (500 μm deep) and the RA (Robust nucleus of the arcopallium which is 2.5 mm deep). Quantification will employ cross-validated metrics of spike stability based on firing pattern that are possible in both areas[27] (FIG. 23). In each case, signal in implanted animals will be sampled with continuous recording for the first month after implant and then for one week intervals for 6 months thereafter. With a large number of recording stations dedicated to this work, these experiments can proceed in parallel, with 12 freely moving birds simultaneously recorded, and 4 mice simultaneously recorded. These experiments are extensions of pilot recordings and the present inventors anticipate no pitfalls. All birds and mice will receive bilateral implants to minimize animal use.

Specific Tests:

a) Splayable, heat-sharpened electrodes, 16, and 32 channels in HVC, and RA. (N=20)

b) Splayable, blunt PEDOT coated electrodes, 16, and 32 channels in HVC, and RA. (N=20)

c) Monolithic blunt PEDOT coated electrodes, 16 and 32 channels in HVC and RA. (N=20)

d) Splayable electrodes, 16, and 32 channels in mouse auditory cortex. (N=10)

Aim 2. Test whether the tunneling arrays are deflected around vasculature.

Histological sections reveal that the microthreads splay during implant (FIG. 23). The hypothesis of the present inventors is that the independent “splayability” of the carbon fibers leads to reduced acute vascular damage relative to a monolithic bundle of fibers that are glued together.

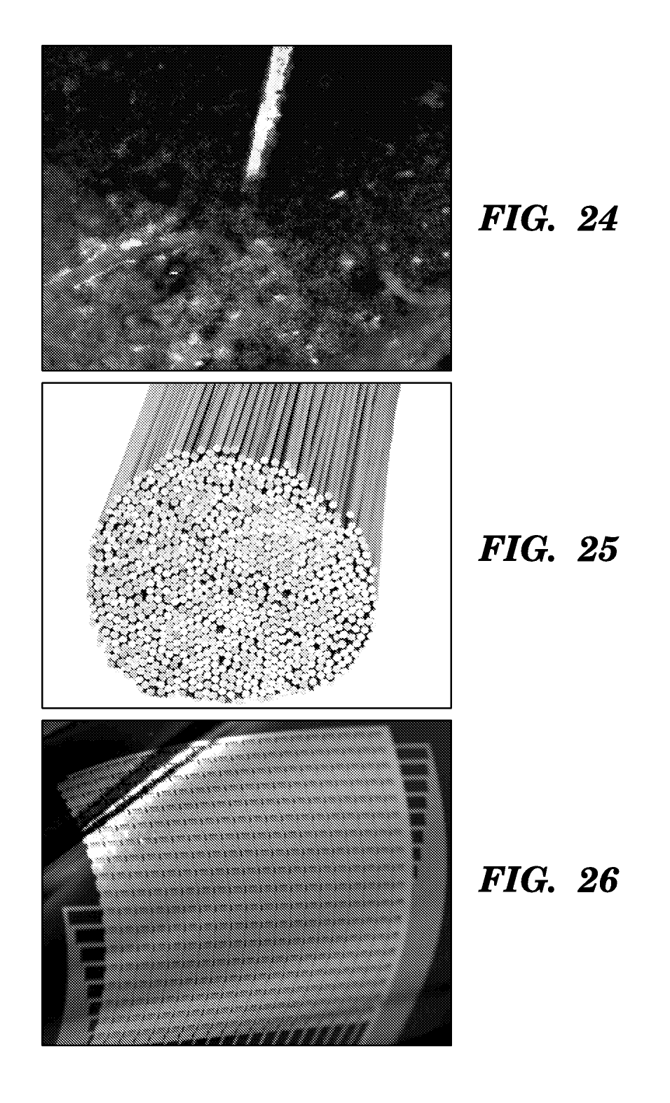

Aim 2 will observe the first 500 μm of the implant process under in-vivo two photon imaging (FIG. 24) of birds and mice whose blood vessels have been labeled with intravenous injection of dye[32] or labeled with quantum dots. The present inventors predict that individual fibers will be deflected by blood vessels, and the present inventors will quantify the extent to which this happens as a function of fiber channel count for 16, 32 and 64 channel arrays. Furthermore, the present inventors will compare this process of tissue penetration for heat-sharpened and blunt cut PEDOT coated tips. (N=15 zebra finches and N=15 mice.)

In a separate group of animals (N=12 zebra finches and N=12 mice) multiple non-functional fiber bundles of size 16, 32, 64 and 128 electrodes fibers will be slowly inserted up to a depth of 5 mm, and secured to the skull. Each animal will receive 4 implants and kept post surgery for another 6-12 hours, allowing early phases of glial activation to set in[35], and then euthanized. The brains will be sectioned in a cryostat perpendicular to the arrays (as in FIG. 24), and stained for markers of glial activation. Antiserum directed against glial fibrillary acidic protein (GFAP) will be used to detect gliosis or activation of astrocytes around the electrodes [36][37], while antiserum directed against CD 45 will be used to detect activated macrophages, and antiserum directed against CD 68 will be used to detect activated microglia[35][38]. The individual tracks of each electrode will be reconstructed through a full series of 50 μm slices, and the magnitude of activated glial staining in proximity to each fiber will be quantified throughout the 5 mm depth of the implant. In N=5 additional songbirds and N=5 mice, the present inventors will analyze blood vessel damage or increased permeability by examining the retention or leakage of dextran conjugated dyes or quantum dots injected into the blood stream[39][40].

If the fibers are observed to diverge around blood vessels during two photon guided implant, and if the individual reconstructed tracks of the fibers in from histological sections are not straight, but bend around blood vessels then the present inventors will infer that in the “tunneling” process fibers may allow each fiber to follow separate paths of least resistance. Control experiments for the histology (N=5 birds, N=5 mice, 4 electrodes per animal) will involve insertion of the same multi-channel carbon fiber electrode, but ensheathed with a second layer of parylene so that the fibers cannot splay.

The experiments will yield statistical information about the extent of the electrode splay as a function of depth in both birds and mice. If the angular divergence of the splaying process is too high, the ability to target defined anatomical locations will be reduced, and designs involving partially glued bundles will be indicated that limit the “splayability” to the final millimeter of electrode length.

If minimally invasive electrode “splaying” can be extended to high channel count (128 channels and above), the result will motivate the development of manufacturing processes such as surface mount connections to multiplexing amplifiers on silicon arrays—designs that would allow for assembly of functional arrays involving hundreds of fibers. The comparison of the extent of splaying in blunt-cut and heat-sharpened electrodes will also inform the design parameters of future arrays.

Expertise to perform the two photon imaging was developed in a previous study by the PI during which time he built a custom two-photon microscope and developed chronic methods for long term two-photon imaging in songbirds[31]. Pilot tests have revealed that the Gardner lab's custom two-photon microscope and imaging protocols are suitable for the proposed acute imaging, and the fluorescent parylene insulation will allow visualization of fiber bundles (seen in low resolution in FIG. 24).

If the electrode bundles are not observed to splay within this field of view, this aim will rely more on the second portion focused on cryosectioning of fixed tissue. Additional possibilities for reconstructing electrode paths relative to vasculature include serial block face imaging or vascular corrosion casting[41] with microCT scanning. The present inventors are currently exploring collaborations to attempt these alternate modes of electrode/vasculature imaging as well.

Aim 3. Test the hypothesis that tunneling fiber arrays are minimally invasive over chronic time-scales.

Birds implanted in the preliminary study were recorded post implant for over one year, and in most cases stable multi-unit signals consisting of a small number of simultaneously recorded cells were stable for months (see FIG. 17 for an example raster pattern that was stable for 100 days). This longevity of recording motivates us to perform a histological study of the chronically implanted electrodes to examine the nature of the interface formed with the tissue. Thanks to a largely hollow skull, zebra finch brains can be cryosectioned without removing the brain from the skull. This allows in-situ electrode histology to be performed without detaching the electrode connector from the skull. Immunocytochemistry and confocal microscopy will be used to analyze the interactions of the electrodes with the brain at a time point of 3 months post implant. Antiserum directed against glial fibrillary acidic protein (GFAP) will be used to detect gliosis or activation of astrocytes around the electrodes[35][36][37], while antiserum directed against CD 45 will be used to detect activated macrophages, and antiserum directed against CD 68 will be used to detect activated microglia[38]. The damage zone of the electrodes will be examined using antiserum directed against the neuronal marker NeuN[42] and the presynaptic marker synaptotagmin. Finally, to examine the extent of ongoing leakage of the blood brain barrier in long term implants, quantum dots (Invitrogen Qtracker Vascular label) will be injected intravenously just prior to perfusion in 5 birds and 5 mice, and extra venous spread quantified.

These measurements will be examined for electrode arrays of 16 and 32 channels, and results cross referenced to the chronic recording data gathered form the same implants (Aim 1). Additional histological comparisons will include monolithic un-splayable electrodes that contain an outer sheath of Parylene, and tunneling array electrodes that are left free floating in the brain (not skull anchored) for the same period of 3 months. The latter comparison will be important to determine if relative motion between the brain and skull leads to chronic tissue damage, even for the sub-cellular scale electrodes used here. This information could inform future attempts to provide anchors for the electrodes in the brain or adhesion molecules that stabilize the connection between electrode and tissue[13]. Paradoxically, it may be the case that larger channel count arrays are more stable in the present design. The surface area of the brain-electrode interface increases linearly with the number of electrodes in the proposed design, and the higher channel counts may adhere more tightly to the tissue. This important question will be directly addressed in the proposed histology and controls. The majority of the long term histology will require no additional animals as it will be performed on the birds mentioned in Aim 1 after chronic recording is completed. This will allow cross referencing of histological results with recording yield and stability. For the tunneling fiber arrays, the present inventors hypothesize that there will be little gliosis, few activated microglia, and that normal neurons and synapses will be in close contact with the electrodes, even for 32 and 64 channel arrays.

Timeline: All aims will begin concurrently employing 16 chronic recording stations for the three month recordings. Long term histology will begin three months after the first chronic implant surgeries. Over a two year time-scale, multiple iterations of each aim can be pursued.

Conclusion: The present inventors anticipate that the present invention will lead to a practical tool for multi-channel stable recording in small animals and beyond. The present invention could be extended to simultaneous cyclic voltammetry, or neural stimulation which may be served by the same carbon-based electrode design.

See FIG. 21. Tunneling microfiber arrays (8 data points along the left side of the chart adjacent the y-axis) have ultra-small minimum feature diameters with high channel count. Cross section is shown in μm.

See FIG. 3, FIG. 4, FIG. 5, FIG. 18A. Single unit recordings of a sparse firing projection neuron and three interneurons. In FIG. 18A, bands reveal boundaries of separate days. Firing patterns are stable.

See FIG. 22. Electrode array (SEM, three length scales left), and single electrode imaged with Anthracene doped parylene (right).

See FIG. 1A, FIG. 1B1, FIG. 1B2. Microthread array assembly.

See FIG. 23. Electrode fibers (white in reverse bright-field) splay over a distance of 300 μm at a depth of 2 mm.

See FIG. 16A. Spike rasters show distinct firing patterns from cells on separate fibers. Firing patterns are stable for one week.

See FIG. 7. Co-variation of spike waveform and firing pattern in HVC can be used to robustly track the longevity of single neuron isolation. Abrupt changes in waveform that coincide with abrupt changes in spike raster pattern signal the end of a period of single unit isolation (top), whereas gradual changes waveform without spike raster changes indicate stable recordings.

See FIG. 24. Two photon in-vivo image of a 16 channel electrode insertion in a transgenic zebra finch. Blood vessels labeled with intravenous dye injection. Electrode cross-section is 25 microns.

See FIG. 17. Stable firing pattern at one site in HVC over a time-scale of 100 days.

The present invention includes a scaled-up version of the embodiments disclosed above. For example, the electrode array can include an increased number of contacts in electrodes, with a goal of ultimately recording from thousands of sites at the same time in the living brain. In preliminary tests, the present inventors have confirmed that carbon fiber arrays can be generalized from the prototype (16-channel) devices to devices consisting of hundreds of independent fibers. When implanting bundles of hundreds of fibers, the implant-splaying process described herein still results in minimal damage to the blood brain barrier at the recording tips of the electrode.

To create functional electrodes comprising hundreds of independent fibers, the current prototype can be modified. It may not be desirable to thread hundreds of fibers through a connector block and utilize silver paint. Instead, a massively large channel count electrode can be formed by any suitable process, including, for example, surface mounting the microthread brush to a two dimensional array of amplifiers.

The design can involve preparing a large bundle of hundreds of electrode fibers, and then gluing the base of the bundle in a solid resin. Cleaving this glued bundle can leave a hexagonal array of electrode ends, as illustrated, for example, in FIG. 25.

To create a functional electrode array, it is necessary to record the voltage on each of the “back ends” of the cleaved electrode. One way of doing this is by surface mounting the cut end of the electrodes to a two-dimensional amplifier array. As shown, for example, in FIG. 26, a two-dimensional array of amplifiers can be formed using any suitable method. The two-dimensional array of amplifiers can be prepared using microelectronics processes on a flexible substrate.

An electrical connection between the bundle and the amplifier can be made by spreading anisotropic conductive paste on the surface and pressing the two parts together. This step eliminates manual assembly, the above-referenced (plastic) block, and silver paint involved in the above-described design.

The main requirement of the present embodiment is that the scale of the array of amplifiers can match the high density of the electrode brush. Current technologies that match this 4-5 micron pitch between fibers include cell phone CCD chips, as well as next generation silicon arrays for neural recording that are expected to be manufactured in the next three years. Tests of this surface mounting approach are underway by the present inventors.

In one embodiment, an electrode array according to the invention can be used for recording and/or stimulation of peripheral nerves, for example, to diagnose and/or treat medical conditions. As described in greater detail above, FIGS. 1C and 22 depict self-splaying microfiber arrays according to the present invention. Specifically, the upper portion of FIG. 1C shows an example of a 16 channel array, and the lower portions of FIG. 1C show examples of Parylene insulation and heat-sharpened or fire-sharpened tips. FIG. 22 shows an example of a bundle of fibers (e.g., held together by Van der Waals forces) and heat-sharpened or fire-sharpened tips, according to some embodiments of the invention. The microfiber arrays of FIGS. 1C and 22 can also be adapted for implantation into and around peripheral nerves for stimulation and/or recording, for example, to diagnose and/or treat medical conditions.

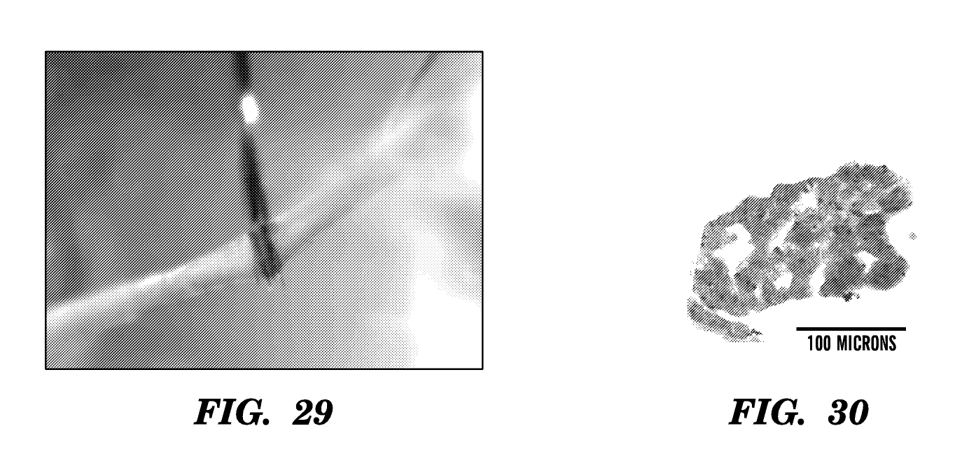

For example, as shown in FIG. 29, self-splaying electrodes such as those illustrated, for example, in any of FIGS. 1, 9, 22, 27 and 28 and described in associated portions of the present specification, are used for recording and stimulation of a songbird hypoglossal nerve tracheo-syringeal (TS) branch. Specifically, FIG. 29 shows sub-micron sharpened electrode tips inserted into a nerve, where the tips splay apart according to the invention. FIG. 30 shows a TS nerve cross-section with an indicator of scale, i.e., 100 microns. That is, the TS nerve has a relatively small diameter. According to some embodiments, the sharpened tips according to the invention can be inserted into small peripheral nerves with little or no consequential damage. As shown, for example, in FIG. 29, an electrode according to the invention, as shown and described herein and above, is inserted into a nerve in a songbird, where the diameter is about 200 microns.

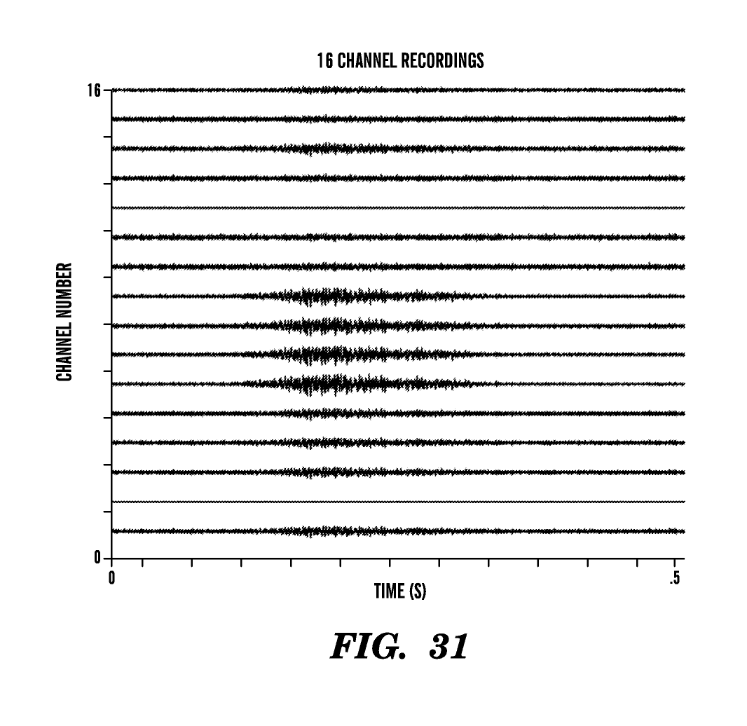

FIG. 31 is a chart depicting 16 channel recordings of self-splaying electrodes in songbird hypoglossal nerve tracheo-syringeal (TS) branch with time (s) on the x-axis from 0 to 0.5 seconds and with the channel number on the y-axis from channel 0 to 16. This electrode can record multiple channels of activity within the relatively small nerve.

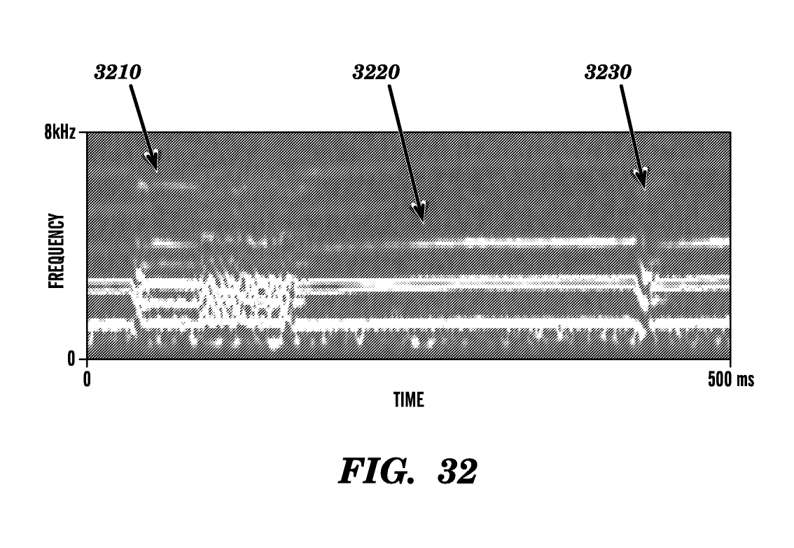

FIG. 32 is a chart depicting vocalizations evoked by TS nerve stimulation in an anesthetized zebra finch. Stimulation 1, identified with arrow 3210, drives a complex vocalization. As identified with arrow 3220, constant pressure airflow through the trachea generates background tone when the nerve is not stimulated. Stimulation 2, identified with arrow 3230, which is located at a different location on the surface of the nerve, evokes a brief frequency modulation. Stimulation patterns 1 and 2 are otherwise identical.

Although some of various drawings illustrate a number of logical stages in a particular order, stages which are not order dependent can be reordered and other stages can be combined or broken out. Alternative orderings and groupings, whether described above or not, can be appropriate or obvious to those of ordinary skill in the art of computer science. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to be limiting to the precise forms disclosed. Many modifications and variations are possible in view of the above teachings. The embodiments were chosen and described in order to best explain the principles of the aspects and its practical applications, to thereby enable others skilled in the art to best utilize the aspects and various embodiments with various modifications as are suited to the particular use contemplated.

Each of the following references, referenced in the description above, is incorporated by reference in its entirety.
Abeles, M., & Goldstein, M. H., Jr. (1977). Multispike train analysis. Presented at the Proceedings of the IEEE.
Angle, M. R., & Schaefer, A. T. (2012). Neuronal recordings with solid-conductor intracellular nanoelectrodes (SCINEs). PLoS ONE, 7(8), e43194-e43194. doi:10.1371/journal.pone.0043194.
Armstrong-James, M., & Millar, J. (1979). Carbon fibre microelectrodes. Journal of Neuroscience Methods, 1(3), 279-287.
Biran, R., Martin, D. C., & Tresco, P. A. (2005). Neuronal cell loss accompanies the brain tissue response to chronically implanted silicon microelectrode arrays. Experimental Neurology, 195(1), 115-126. doi:10.1016/j.expneuro1.2005.04.020.
Chestek, C. A., Batista, A. P., Santhanam, G., Yu, B. M., Afshar, A., Cunningham, J. P., Gilja, V., et al. (2007). Single-neuron stability during repeated reaching in macaque premotor cortex. Journal of Neuroscience, 27(40), 10742-10750. doi:10.1523/JNEUROSCI.0959-07.2007.
Chorover, S. L., & DeLuca, A. M. (1972). A sweet new multiple electrode for chronic single unit recording in moving animals. Physiology & behavior, 9(4), 671-674.
Cortes, C., & Vapnik, V. (1995). Support-vector networks. Machine learning. 20 273-97.
Dempster, A. P., Laird, N. M., & Rubin, D. B. (1977). Maximum likelihood from incomplete data via the EM algorithm. Journal of the Royal Statistical Society. Series B (Methodological), 1-38.
Dickey, A. S., Suminski, A., Amit, Y., & Hatsopoulos, N. G. (2009). Single-unit stability using chronically implanted multielectrode arrays. Journal of Neurophysiology, 102(2), 1331-1339. doi:10.1152/jn.90920.2008.
Donoghue, J. P. (2008). Bridging the brain to the world: a perspective on neural interface systems. Neuron, 60(3), 511-521. doi:10.1016/j.neuron.2008.10.037.
Fee, M. S., & Leonardo, A. (2001). Miniature motorized microdrive and commutator system for chronic neural recording in small animals. Journal of Neuroscience Methods, 112(2), 83-94.
Fraser, G. W., & Schwartz, A. B. (2012). Recording from the same neurons chronically in motor cortex. Journal of Neurophysiology, 107(7), 1970-1978. doi:10.1152/jn.01012.2010.
Garris, P. A., Ciolkowski, E. L., Pastore, P., & Wightman, R. M. (1994). Efflux of dopamine from the synaptic cleft in the nucleus accumbens of the rat brain. The Journal of neuroscience: the official journal of the Society for Neuroscience, 14(10), 6084-6093.
Gray, C. M., Maldonado, P. E., Wilson, M., & McNaughton, B. (1995). Tetrodes markedly improve the reliability and yield of multiple single-unit isolation from multi-unit recordings in cat striate cortex. Journal of Neuroscience Methods, 63(1-2), 43-54. doi:10.1016/0165-0270(95)00085-2.
Hahnloser, R. H. R., Kozhevnikov, A. A., & Fee, M. S. (2002a). An ultra-sparse code underlies the generation of neural sequences in a songbird. Nature, 419(6902), 65-70. doi:10.1038/nature00974.
Hahnloser, R. H. R., Kozhevnikov, A. A., & Fee, M. S. (2002b). An ultra-sparse code underlies the generation of neural sequences in a songbird. Nature, 1-7.
Harvey, C. D. C., Coen, P. P., & Tank, D. W. D. (2012). Choice-specific sequences in parietal cortex during a virtual-navigation decision task. Nature, 484(7392), 62-68.
Jones, K. E., Campbell, P. K., & Normann, R. A. (1992). A glass/silicon composite intracortical electrode array. Annals of biomedical engineering, 20(4), 423-437.
Katz, L. C., & Gurney, M. E. (1981). Auditory responses in the zebra finch's motor system for song. Brain Research, 221(1), 192-197.
Keefer, E. W., Botterman, B. R., Romero, M. I., Rossi, A. F., & Gross, G. W. (2008). Carbon nanotube coating improves neuronal recordings. Nature nanotechnology, 3(7), 434-439. doi:10.1038/nnano.2008.174.
Kentros, C. G., Agnihotri, N. T., Streater, S., Hawkins, R. D., & Kandel, E. R. (2004). Increased attention to spatial context increases both place field stability and spatial memory. Neuron, 42(2), 283-295.
Kim, D.-H., Viventi, J., Amsden, J. J., Xiao, J., Vigeland, L., Kim, Y.-S., Blanco, J. A., et al. (2010). Dissolvable films of silk fibroin for ultrathin conformal bio-integrated electronics. Nature materials, 9(6), 511-517. doi:10.1038/nmat2745.
Koralek, A. C., Jin, X., Long, J. D., II, Costa, R. M., & Carmena, J. M. (2012). Corticostriatal plasticity is necessary for learning intentional neuroprosthetic skills. Nature, 483(7389), 331-335. doi:10.1038/nature10845.
Kozai, T. D. T. Y., Langhals, N. B. N., Patel, P. R. P., Deng, X. X., Zhang, H. H., Smith, K. L. K., Lahann, J. J., et al. (2012). Ultrasmall implantable composite microelectrodes with bioactive surfaces for chronic neural interfaces. Nature materials, 11(12), 1065-1073. doi:10.1038/nmat3468.
Kozai, T. K., LANGHALS, N. B., Patel, P. R., Deng, X., Zhang, H., Lahann, J., Kotov, N. A., et al. (2010). In vivo chronic cortical recordings using novel ultra-small carbon fiber based implantable microthread ultramicroelectrodes. Presented at the 2010 Neuroscience Meeting Planner. San Diego, Calif.: Society for Neuroscience, 2010. Online.
Kozhevnikov, A. A., & Fee, M. S. (2007). Singing-Related Activity of Identified HVC Neurons in the Zebra Finch. Journal of Neurophysiology, 97(6), 4271-4283. doi:10.1152/jn.00952.2006.
Leonardo, A., & Fee, M. S. (2005). Ensemble coding of vocal control in birdsong. Journal of Neuroscience, 25(3), 652-661. doi:10.1523/JNEUROSCI.3036-04.2005.
Lewicki, M. S. (1996). Intracellular characterization of song-specific neurons in the zebra finch auditory forebrain. The Journal of neuroscience: the official journal of the Society for Neuroscience, 16(18), 5855-5863.
Lin, J. (1991). Divergence measures based on the Shannon entropy. Information Theory. Long, M. A., Jin, D. Z., & Fee, M. S. (2010). Support for a synaptic chain model of neuronal sequence generation. Nature, 468(7322), 394-399. doi:10.1038/nature09514.
Ludwig, K. A., Miriani, R. M., LANGHALS, N. B., Joseph, M. D., Anderson, D. J., & Kipke, D. R. (2009). Using a Common Average Reference to Improve Cortical Neuron Recordings From Microelectrode Arrays. Journal of Neurophysiology, 101(3), 1679-1689. doi:10.1152/jn.90989.2008.
Mooney, R. (2005). The HVC Microcircuit: The Synaptic Basis for Interactions between Song Motor and Vocal Plasticity Pathways. Journal of Neuroscience, 25(8), 1952-1964. doi:10.1523/JNEUROSCI.3726-04.2005.
Nicolelis, M. A., Lehew, G., & Nicolelis, M. A. L. (2008). State-of-the-Art Microwire Array Design for Chronic Neural Recordings in Behaving Animals (2nd ed.). Boca Raton (Fla.): CRC Press.
Polikov, V. S., Tresco, P. A., & Reichert, W. M. (2005). Response of brain tissue to chronically implanted neural electrodes. Journal of Neuroscience Methods, 148(1), 1-18. doi:10.1016/j.jneumeth.2005.08.015.
Poole, B. B., Markowitz, J. E. J., & Gardner, T. J. T. (2012). The song must go on: resilience of the songbird vocal motor pathway. PLoS ONE, 7(6), e38173-e38173. doi:10.1371/journal.pone.0038173.
Quiroga, R. Q., Nadasdy, Z., & Ben-Shaul, Y. (2004). Unsupervised spike detection and sorting with wavelets and superparamagnetic clustering. Neural computation, 16(8), 1661-1687. doi:10.1162/089976604774201631
Rissanen, J. (1978). Modeling by shortest data description. Automatica.
Rokni, U., Richardson, A. G., Bizzi, E., & Seung, H. S. (2007). Motor learning with unstable neural representations. Neuron, 54(4), 653-666. doi:10.1016/j.neuron.2007.04.030.
Schmitzer-Torbert, N., Jackson, J., Henze, D., Harris, K., & Redish, A. D. (2005). Quantitative measures of cluster quality for use in extracellular recordings. Neuroscience, 131(1), 1-11. doi:10.1016/j.neuroscience.2004.09.066.
Schulte, A., & Chow, R. H. (1998). Cylindrically etched carbon-fiber microelectrodes for low-noise amperometric recording of cellular secretion. Analytical chemistry, 70(5), 985-990. doi:10.1021/ac970934e.
Scott, B. B., Gardner, T., Ji, N., Fee, M. S., & Lois, C. (2012). Wandering Neuronal Migration in the Postnatal Vertebrate Forebrain. Journal of Neuroscience.
Subbaroyan, J., Martin, D. C., & Kipke, D. R. (2005). A finite-element model of the mechanical effects of implantable microelectrodes in the cerebral cortex. Journal of Neural Engineering, 2(4), 103-113. doi:10.1088/1741-2560/2/4/006.
Thompson, L. T., & Best, P. J. (1990). Long-term stability of the place-field activity of single units recorded from the dorsal hippocampus of freely behaving rats. Brain Research, 509(2), 299-308.
Tolias, A. S., Ecker, A. S., Siapas, A. G., Hoenselaar, A., Keliris, G. A., & Logothetis, N. K. (2007). Recording chronically from the same neurons in awake, behaving primates. Journal of Neurophysiology, 98(6), 3780-3790. doi:10.1152/jn.00260.2007.

1. An electrode array comprising:
a bundle of individually addressable, insulated micro-fibers with uninsulated, exposed tips, wherein the bundle of micro-fibers splay apart upon implantation.

2.-45. (canceled)

46. The electrode array of claim 1, wherein the micro-fibers comprise carbon.

47. The electrode array of claim 1, wherein the micro-fibers are held together by van der Waals forces, and splay apart upon implantation.

48. The electrode array of claim 1, wherein the micro-fibers comprise a conductive, memoryless material having material properties amenable to splaying during implantation.

49. The electrode of claim 1, wherein the exposed tips are sharpened by heating.

50. The electrode of claim 1, wherein the exposed tips are prepared by blunt cutting or by use of a focused ion beam.

51. The electrode array of claim 1, wherein the tips are heat-sharpened at an air-liquid interface.

52. The electrode array of claim 1 comprising:
a micro-channel block comprising an opening extending through the block,
wherein the micro-fibers extend through the opening.

53. The electrode array of claim 52, wherein the block comprises plastic or another machineable material.

54. The electrode array of claim 52, wherein the block is formed by a 3D printing process.

55. The electrode array of claim 52, wherein the block comprises:
a main body;
a pair of arms extending from the main body; and
a funnel suspended by the pair of arms,
wherein the micro-fibers pass through the funnel.

56. The electrode array of claim 55, wherein the funnel comprises an aperture having a diameter in a range from 100 microns to 500 microns.

57. The electrode array of claim 1, wherein each of the micro-fibers has a diameter of about 3-10 microns.

58. The electrode array of claim 57, wherein the diameter of each electrode is about 4.5 microns.

59. The electrode array of claim 1, wherein the insulated micro-fibers are insulated with parylene deposited on each of the micro-fibers at a thickness of about 1-3 microns.

60. The electrode array of claim 52, wherein the opening is filled with a conductive material to provide electrical contact between the micro-fibers and an electrical connector.

61. The electrode array of claim 1, wherein the tips are heat-sharpened with a gas/oxygen torch.

62. The electrode array of claim 61, wherein the impedance of the heat-sharpened tips is in a range of 0.1-1.5 MΩ.

63. The electrode array of claim 62, wherein the average impedance is about 1.2 MΩ.

64. The electrode array of claim 1, wherein the bundle of micro-fibers has an overall diameter of about 26 microns for a 16-channel device, about 36 microns for a 32-channel device, and about 50 microns for a 64-channel device.

65. The electrode array of claim 1, wherein each of the micro-fibers has an exposed tip having a length in the range from 72 microns to 106 microns.

66. The electrode array of claim 65, wherein the length is about 89 microns.

67. The electrode array of claim 1, wherein the bundle of micro-fibers is adapted to splay during implantation into a subject.

68. The electrode array of claim 1, wherein the electrode array yields stable signals over a time period of greater than a week.

69. The electrode array of claim 68, wherein the time period is greater than a month.

70. A method of manufacturing an electrode array comprising:
bundling a plurality of individually addressable, insulated micro-fibers; and
exposing a tip of each of the plurality of insulated micro-fibers by heat-sharpening at an air-liquid interface to remove the insulation.

71. The method of manufacturing an electrode array of claim 70, wherein the micro-fibers comprise carbon.

72. The method of manufacturing an electrode array of claim 70, wherein the micro-fibers are held together by van der Waals forces, and splay apart upon implantation.

73. The method of manufacturing an electrode array of claim 70, wherein the micro-fibers comprise a conductive, memoryless material having material properties amenable to splaying during implant.

74. The method of manufacturing an electrode array of claim 70 comprising:
lowering the electrode array into a liquid bath with tips of the plurality of fibers protruding above a surface of the liquid bath; and
passing a heating means over the surface of the liquid bath thus burning the plurality of fibers down to a surface of the liquid bath and forming an uninsulated, sharpened tip from each of the plurality of fibers.

75. The method of manufacturing an electrode array of claim 70 comprising:
raising the electrode array from a liquid bath with the tips of the plurality of fibers initially pointing down into the liquid bath; and
bundling the plurality of fibers with surface tension acting on the plurality of fibers as the electrode array is removed from the liquid bath.

76. The method of manufacturing an electrode array of claim 70 comprising:
filling the plurality of openings with a conductive material.

77. The method of manufacturing an electrode array of claim 70 comprising:
forming a block comprising a plurality of openings through the block; and
threading each of the plurality of fibers through each of the plurality openings in the block.

78. The method of manufacturing an electrode array of claim 77 comprising:
passing the plurality of fibers through a funnel suspended from a main body of the block in order to bundle the plurality of fibers.

79. The method of manufacturing an electrode array of claim 74, wherein micro-fibers of multiple lengths are prepared by holding the electrode array at an angle relative to a liquid surface during the heat-sharpening process.

80. A method of implanting an electrode array into a subject, the electrode array comprising a splayable bundle of individually addressable, insulated micro-fibers with uninsulated, exposed tips, the method comprising:
exposing a target area in the subject for the electrode array; and
inserting the bundle of micro-fibers into the target area,
wherein forces holding the bundle of micro-fibers together are released during the insertion, resulting in micro-fibers splaying as they move into the target area.

81. The method of claim 80, wherein the micro-fibers comprise carbon.

82. The method of claim 80, wherein the micro-fibers are held together by van der Waals forces, and the van der Waals forces are released causing the micro-fibers to splay apart upon insertion.

83. The method of claim 80, wherein the micro-fibers comprise a conductive, memoryless material having material properties amenable to splaying during implant.

84. The method of claim 80, wherein the micro-fibers splay over a distance of about 300 μm at a depth of about 2 mm into the subject.

85. The method of claim 80, wherein a degree of splaying is increased by a lateral tension held in the micro-fibers during the inserting step.

86. The method of claim 80, wherein a degree of splaying is limited by partially gluing micro-fibers together before the inserting step, allowing an end of the bundle to splay while a body of the bundle does not splay.

87. The method of claim 80, wherein a geometry of the splayed array is controlled by preparing micro-fibers of multiple lengths in a single bundle.
